(19) United States
(12) Patent Application Publication
(10) Pub. No.: US 2017/0225986 A1
(43) Pub. Date: Aug. 10, 2017

(54) BIOFILM PROCESS FOR TREATING WATER WITH CONTINUOUS OR SEMI-CONTINUOUS PRODUCTION OF BIOMASS WITH ENHANCED POLYHYDROXYALKANOATE CONTENT

(71) Applicant: VEOLIA WATER SOLUTIONS & TECHNOLOGIES SUPPORT, SAINT MAURICE (FR)

(72) Inventors: Alan Gideon Werker, Lomma (SE); Lamija Karabegovic, Helsingborg (SE); Luca Markus Quadri, Malmo (SE); Monica Vanessa Arcos Hernandez, Nezahualcoyotl, Estado de Mexico (MX); Carl Anton Borje Karlsson, Asa (SE)

(73) Assignee: VEOLIA WATER SOLUTIONS & TECHNOLOGIES SUPPORT, SAINT MAURICE (FR)

(21) Appl. No.: 15/501,515

(22) PCT Filed: Jul. 31, 2015
(86) PCT No.: PCT/IB2015/055839

§ 371 Date: Feb. 3, 2017

Related U.S. Application Data
(60) Provisional application No. 62/032,701, filed on Aug. 4, 2014.

(51) Int. Cl. C02F 3/08; C12P 7/62; C02F 3/34; C02F 3/00; C02F 3/10

(52) U.S. Cl. CPC C02F 3/085; C02F 3/006; C02F 3/109; C02F 3/341; C12P 7/625; C02F 2103/28

(57) ABSTRACT

A biofilm process is disclosed for treating wastewater containing readily biodegradable dissolved organic matter GP (measured as chemical oxygen demand or COD) and producing surplus biomass from the biofilm process that includes an enhanced polyhydroxyalkanoate (PHA) content. The process comprises directing a wastewater influent containing the readily biodegradable COD (RBCOD) into a biofilm unit process. The PHA content of surplus biomass is enhanced by controlling for a decreased biofilm process specific organic loading rate in combination with controlling phosphorus loading rates relative to the process RBCOD loading rates: (1) controlling the wastewater influent phosphorus loading rate to the biofilm unit process includes maintaining an average RBCOD/P ratio of the influent that is between 200 and 800 g/g; (2) decreasing the process specific organic loading rate includes producing a biofilm unit process effluent having readily separable mixed liquor volatile suspended solids (RS-MLVSS); and (3) separating a portion of the RS-MLVSS from the biofilm unit process effluent and recycling at least a portion of the separated RS-MLVSS back to the biofilm unit process. The combination of the RBCOD/P control and specific loading rate control maintains, on average, the surplus biomass with a PHA content that is greater than 30% gPHA/g VSS.

### RELATED APPLICATIONS

This application claims priority under 35 U.S.C. §119(e) from the following U.S. provisional application: Application Ser. No. 62/032,701 filed on Aug. 4, 2014. That application is incorporated in its entirety by reference herein.

### FIELD OF THE INVENTION

The methods disclosed herein relate to biologically treating wastewater streams and accumulating polyhydroxyalkanoate (PHA).

### BACKGROUND OF THE INVENTION

Biological treatment of industrial process waters and wastewaters for removal of dissolved organic contamination measured as chemical oxygen demand (COD) produces a by-product in the form of an excess biomass. Such excess, or so-called surplus, biomass is commonly regarded as a solid waste disposal problem. However, the burden of this surplus biomass management can be transformed into an opportunity when this by-product can be exploited as a resource instead of being a waste. Towards this end, a biomass that is able to accumulate significant amounts of intracellular storage products as biopolymers, in the form of PHAs, can be used as a resource in a commercial bioplastics value chain. Thus, there is a need to develop efficient methods for accumulating PHAs of sufficient quality for use in commercial bioplastic products.

### SUMMARY OF THE INVENTION

In one embodiment, the process disclosed herein comprises a biofilm process for treating a wastewater stream containing RBCOD and producing biomass with enhanced PHA content from the biofilm process. The process includes directing a wastewater influent containing the RBCOD into a biofilm unit process and enhancing the PHA content of detached or surplus biomass by: (1) controlling the wastewater influent to the biofilm unit process to maintain an average RBCOD/P ratio of between 200-800 g/g; (2) producing a biofilm unit process effluent having readily separable mixed liquor volatile suspended solids (RS-MLVSS); and (3) separating a portion of the RS-MLVSS from the biofilm unit process effluent and recycling at least a portion of the separated RS-MLVSS back to the biofilm unit process. A combination of the RBCOD/P control and the RS-MLVSS circulation maintains on average the surplus biofilm with a PHA content that is greater than 30% gPHA/gVSS.

In another embodiment, a similar biofilm process is described for enhancing the PHA content in surplus or detached biomass. Here at least a fraction of the mixed liquor volatile suspended solids (MLVSS) are separated from the biofilm unit process effluent and recycled back to the biofilm unit process such that a process average MLVSS concentration is at least 10% higher than an average MLVSS concentration for the same process and operating conditions but without any MLVSS recycling. Again, the combination of the RBCOD/P control and the MLVSS recirculation maintains, on average, the surplus biomass with a PHA content that is greater than 30% gPHA/gVSS.

In yet another embodiment, there is disclosed a moving bed biofilm reactor (MBBR) process for treating a wastewater stream containing RBCOD and producing a biomass with enhanced PHA content within the MBBR process. This process entails directing a wastewater influent containing the RBCOD into the MBBR unit process and enhancing the PHA content in a surplus biomass by: (1) controlling the wastewater influent being directed to the MBBR unit process to maintain an average RBCOD/P ratio of between 200-800 g/g; (2) producing an MBBR unit process effluent having MLVSS; and (3) separating at least a fraction of the MLVSS from the MBBR unit process effluent and recycling the fraction of separated MLVSS back to the MBBR unit process such that a process average MLVSS concentration is at least 10% higher than an average MLVSS concentration for the same process and operation conditions but without any MLVSS recycling. Similar to the other embodiments described above, the combination of the RBCOD/P control and the MLVSS recirculation maintains, on average, the surplus biomass with a PHA content that is greater than 30% gPHA/gVSS.

### DESCRIPTION OF THE DRAWINGS

FIG. 1 is a graph showing biological treatment performance in RBCOD removal and surplus biomass PHA content as a function of RBCOD/P for Paper Mill (Left) and Reference (Right) influent.

FIG. 2 is a graph showing observations of selected RBCOD/N on process performance for PHA production and RBCOD removal (Paper Mill influent).

FIG. 3 is a graph showing observations of selected RBCOD/N on process performance for PHA production and RBCOD removal (Reference influent).

FIG. 4 is a graph showing the observed performance of PHA production (excess biomass PHA content) with thermophilic operating conditions (50° C.).

FIG. 5 is a graph showing an observed influence of process background RBCOD on PHA production.

FIG. 6 is a graph showing the average increased performance of continuous 1-step PHA production through increase of the process biofilm bio-volume capacity.

FIG. 7 is a graph showing the influence of an increase in process biofilm biomass as represented by an indicative carrier specific biofilm bio-volume content as a function of biocarrier filling degree.

FIG. 8 is a graph showing, with reference to FIG. 7, respective changes in performance of RBCOD removal as influenced by changes in the indicative carrier specific biofilm bio-volume content as a function of biocarrier filling degree.

FIG. 9 depicts a schematic showing an illustrative process train for the continuous or semi-continuous production of PHA as part of treatment of a process water or wastewater.

FIG. 10 depicts a schematic showing an illustrative unit process for continuous or semi-continuous MBBR PHA production, with reference to item 40 and the process train illustrated in FIG. 9.

FIG. 11 depicts a photograph of an AnoxKaldnes Z-MBBR biocarrier element with picture taken on a reflective surface.

FIG. 12 depicts a photograph of an AnoxKaldnes Z-MBBR biocarrier element filled to biofilm capacity with picture taken on a reflective surface.

FIG. 13 depicts a graph showing the average PHA content of the effluent MLVSS biomass from the R- and D-MBBRs over the period of balanced P (RBCOD/P=200 g/g) and with a small shift to limiting P-loading (RBCOD=326 and 266 g/g for the R-MBBR and D-MBBR, respectively).

FIG. 14 depicts a graph showing the average PHA accumulation potential (PAP) of the effluent MLVSS biomass from the R- and D-MBBRs over the period of balanced P (RBCOD/P=200 g/g) and with a small shift to limiting P-loading (RBCOD=326 and 266 g/g for the R-MBBR and D-MBBR, respectively).

FIG. 15 depicts a graph showing the average effluent MLVSS biomass PHA content from the R- and E-MBBRs comparing periods of applied RBCOD/P equal to 310, 348 and 398 g/g.

### DETAILED DESCRIPTION OF THE INVENTION

PHAs are biopolymers that can be recovered from a PHA-rich-biomass and converted into biodegradable plastics of commercial value within an increasingly broadening range of practical applications. It has been shown that mixed culture processes such as activated sludge may be engineered to produce a biomass with significant PHA accumulating potential. This biomass can be made to accumulate PHA of high molecular weight when fed with readily biodegradable COD (RBCOD). A PHA-rich-biomass can be processed to enable the recovery of these value added polymers wherein the polymers are with high thermal stability. Examples of the process elements to produce biomass, accumulate PHA, and manage the PHA-rich-biomass for polymer recovery may be found discussed by examples in US 2010/0200498, WO 2011/070544A2, WO 2011/073744A1, WO 2012/022998A1, and WO 2012/023114A1. Therefore, services of water quality improvement may be coupled to a purpose of biomass production, wherein this biomass is made to contain, to a significant fraction of its total mass, polymers or co-polymer blends of PHA that may be subsequently recovered and utilized as a resource.

It is known that PHAs are generally stored as intracellular granules by many species of bacteria. These bacteria store PHA when they are submitted to stress conditions caused by limitation of a nutrient but are otherwise with supply of an organic carbon source. Biomass coming from wastewater treatment plants (VWVTPs) such as activated sludge is a mixed culture generally comprised of a number of populations of species of microorganisms. The species that comprise the microbial populations may be generally unknown specifically. Notwithstanding, for our purposes, we consider a biomass to be enriched with phenotypic PHA storing bacteria when the functional ability to store PHA can be demonstrated by, for example, feeding such biomass with RBCOD such as volatile fatty acids. We consider that a biomass is enriched with populations of species of PHA-storing bacteria when the PHA accumulation potential (PAP) reaches or exceeds 30% gPHA/gVSS. Enrichment of a biomass with the PHA storing phenotype may further be shown qualitatively or even quantitatively by staining methods such as with Nile Red, wherein the stain can indicate by microscopy the general distribution of bacteria in the biomass that participate in PHA storing metabolic activity.

The maximum extant PHA accumulation potential of a biomass will depend in part on the level of enrichment with the PHA-storing phenotype. It will also depend on the biomass physiological state (or history) in advance of a PHA accumulation process. Finally the PHA accumulation potential may also be influenced by the technology and methods in approach taken to stimulate a biomass to accumulate PHA. For improved economy in PHA recovery from biomass, it is generally preferable to produce a biomass with PHA content in excess of 40% and more preferably in excess of 50% gPHA/gVSS. Therefore, the PHA accumulation potential (PAP) of the biomass should be in excess of 40% and more preferably in excess of 50% gPHA/gVSS. It is desirable to produce a biomass such that it expresses as much of its extant PAP as possible.

A distinction is to be made between metabolic processes of biomass growth and PHA storage. Both processes result in a net increase in the amount of biomass. Biomass growth refers to an increase in the number of active bacterial cells per unit bioprocess volume. Biomass growth is due to bacterial cell division and the general proliferation and increase in the number of bacteria that comprise the biomass. PHA storage, or PHA production, is due to the accumulation of intracellular granules of PHA within the PHA storing bacteria that comprise the biomass. The amount of biomass may also become increased, for example, due to production of extra-cellular material and/or an increase of individual bacterial cell size in absence of cell division. Notwithstanding, we consider biomass growth to be a general increase (per unit volume) in the active biomass and estimate changes in this active biomass by changes in the difference between the total measured amount of biomass (per unit volume) less the amount of PHA mass (within the same unit volume). We consider biomass storage to refer to the intracellular storage of PHA in the biomass and we measure storage by an increase in the content of PHA within a biomass or by the increase in the amount of PHA (per unit volume) in a bioprocess.

Microbial metabolism for PHA storage as well as biomass growth may in general be aerobic or anoxic. Therefore, the methods and process presented herein for continuous or semi-continuous PHA production embody both aerobic and anoxic biological processes that treat RBCOD from process waters and wastewaters. Process waters are a by-product of an industrial process and may, in part, be recycled back within the industrial process after treatment. An example is a paper mill where the water used for pulping and paper making may be used and re-used in the process while as part of the process there is a step to remove some contamination from the process water so that it may be reused again. Wastewater is a water effluent produced as waste from municipal or industrial activity. It is an effluent that is to be discharged to the receiving environment after some form of treatment. As both types of waters may be applicable to the methods and processes described herein, we use the expression wastewater without limitation to refer to any influent water to a process, in general, that requires treatment for some form of water quality improvement within the said process.

Specific selection and enrichment for the phenotype of PHA accumulating bacteria within a biomass is generally accomplished by choosing operational conditions in the bioprocess that give a preference for survival in the biomass of those species of bacteria that can store PHA. It is generally known to experts in the field, for example, that one means to enrich an activated sludge with the potential to accumulate PHA while treating a wastewater, is to apply a so-called dynamic feeding, or feast-famine feeding, strategy. Cycling a biomass through periods of feast (RBCOD availability) and famine (RBCOD scarcity) will tend to enrich the biomass with the presence of the PHA storing phenotype within the surplus biomass. When bacteria are submitted to this repeated alternation between feast and famine environments, those bacteria which can assimilate the supplied RBCOD more rapidly as stored PHA, and then utilize that intracellular stored PHA as a carbon and energy reserve during famine, gain a selective advantage over other non-PHA storing bacteria. Thus PHA storage may provide some bacteria with a kinetic advantage to sequester carbon more rapidly than other non-PHA storing bacteria.

During feast, at least some RBCOD is generally converted into PHA. Under famine conditions with low RBCOD availability, the biomass disposed from conditions of feast to conditions of famine may use any internally stored PHA as a source of energy and carbon to sustain general maintenance needs and for growth. Feast is characterized by a relatively high biomass respiration rate, and famine is characterized by a relatively low or endogenous biomass respiration rate. Those populations of species of bacteria, which tend to grow the most, become more dominate in the surplus biomass and these more successful species of bacteria are said to become selected, or enriched, in the biomass. Notwithstanding, the word selected in the present application refers not to specific species of bacteria but to enhancement of function due to increased presence of those populations of species of bacteria, in general, that express the phenotypic behaviour of PHA storage metabolism.

When using methods and process of PHA production from mixed cultures (such as activated sludge), it is common to separate the polymer production into two distinct process steps, namely, Step 1: Biomass Production, and Step 2: PHA Production. During biomass production (Step 1), conditions of enrichment, such as repeated cycles of feast and famine, are provided so as to select for a biomass exhibiting a high extant PHA accumulation potential (see for example WO 2011/073744A1). In subsequent PHA production (Step 2), surplus biomass coming from Step 1 is made to accumulate PHA to a maximal possible extent within a well-controlled bioprocess that imposes conditions of a sustained period of feast on the biomass coming from Step 1 (see for example WO 2011/070544A2).

The PHA production process (Step 2) may be considered to be a process of sustained feast because RBCOD is supplied continuously or semi-continuously, to the process mixed liquor containing the process biomass. Over a sustained period of time the PHA content of that biomass increases from initial low levels, that are generally less 5% of the biomass measured as volatile suspended solids or VSS (gPHA/gVSS). Given successful selection methods in Step 1, the PHA content for an enriched biomass may increase in Step 2 to significantly high levels that are generally more than 30% but preferably greater than 40% (gPHA/gVSS). Ideally, Step 2 should strive to permit the biomass to accumulate PHA up to the extant level of the biomass PAP.

If one or more nutrients that are required for biomass growth are limiting, the capacity for PHA storage is reached and the respiration of the biomass diminishes significantly in time during Step 2 because the biomass cannot store more PHA and the bacteria lack some essential nutrient to be able to utilize and grow on the intracellular stored material. In such an example, the PHA production (Step 2) is a batch-process wherein the Step 2 end-point occurs as the biomass reaches a maximum extant possible PHA content, and the respiration response to any further supplied RBCOD decreases significantly. This decrease in maximum extant respiration rate signifies that any additional supply of RBCOD will not become utilized by the biomass. The fed-batch process can be terminated; the PHA-rich-biomass may then be collected and disposed to further steps of dewatering in preparation for the polymer recovery (see for example: WO 2012/022998A1).

Notwithstanding, when nutrients are also provided with RBCOD, the biomass may metabolize some of the supplied organic matter for growth as well as for purposes of PHA storage. We found that the accumulation process may be prolonged and the process productivity may be increased by using selected blends of RBCOD, nitrogen and phosphorus in the feedstock used for a (Step 2) PHA production process (see PCT/162014/058242). Thus we found that an enriched biomass could be sustained to grow while also storing PHA meaning that the fed-batch process may be prolonged in duration by a period of steady state where there is no net increase in the PHA content of the biomass, but the mass of PHA-rich-biomass increases steadily in time due to concurrent active biomass growth and PHA storage (Valentino et al. (2015). Water Research. 77, 49-63). Therefore, when sufficient nutrients are provided in Step 2, PHA-rich-biomass may be harvested not just at the end of the fed-batch process but also in takt with the net increase of total PHA-rich-biomass during this period of observed steady state PHA-content.

Based on these observations wherein we found a period of steady-state of both active biomass and PHA production during a fed-batch Step 2, we sought to extend this development to a continuous PHA production process for which we could combine the principles of biomass selection (Step 1) and PHA production (Step 2) into a single one-step bioprocess. We have consequently discovered methods and process for a novel single step biofilm open culture process whereby selection conditions are imposed to enrich and maintain for the PHA storing phenotype all the while that a PHA-rich surplus biomass is produced. Further, the operating conditions of the bioprocess are selected so as to stimulate an enhancement of PHA content in the surplus biomass of more than 30% gPHA/gVSS.

Without limitation, we have made these subsequent discoveries based on the operation of a biofilm process, and more particularly with the use of a moving bed biofilm reactor (MBBR). Such biofilm processes may be generally applied in services of water quality improvement for industrial process waters and wastewaters. Biofilms are commonly used in bioprocesses as a means to decouple the so-called hydraulic residence time (HRT) from the so-called solids residence time (SRT). The HRT is the average time that an influent water molecule is present in the bioprocess before it ends up in the process effluent. The SRT is the average time a particle of biomass (or a given bacterial cell) is present in the bioprocess before it exits the bioprocess as surplus (produced) biomass.

By maintaining an overall process biomass SRT that is longer than the HRT, one may increase the resident total mass of biomass in the process and this may provide for an increased rate of RBCOD removal per unit bioreactor liquid volume. Those skilled in the art understand that in a biofilm process, the principal inventory of biomass in the process is generally maintained in the process due to biomass attachment on the available surfaces that are provided for. Notwithstanding the history in development and knowledge of biofilm wastewater treatment systems, methods to continuously or semi-continuously produce a biomass in the effluent of a biofilm process with enhanced PHA content have not been previously described.

We find that one preferred embodiment for continuously or semi-continuously producing a PHA-rich biomass is with biofilm processes where the overall total biomass SRT, namely for the principal biofilm biomass, is inherently longer than the HRT. Here, the dominant inventory of biomass is maintained as a biofilm, wherein suspended biomass in the process mixed liquor is also dominantly a product of biomass growth within a biofilm and detachment from the process biofilm. Without limitation to the type of biofilm process, we present the preferred embodiments by the example of a moving bed biofilm process, wherein the effluent biomass suspended solids are predominantly derived and produced from the process biofilm, and that these biofilm solids that enter into the process mixed liquor and are separated from the process effluent, are produced with enhanced PHA content.

A Moving Bed Biofilm Reactor (MBBR) is a biofilm process where biofilm support elements are mixed within the reactor volume mixed-liquor and a biofilm biomass may grow on the surface area supplied by virtue of the presence of these support elements. Support elements may be made, for example, of plastic or rubber, but sand particles can also be viable biofilm support elements. Without limitation we refer to these support elements as (biofilm) carriers or biocarriers in the biofilm process.

Biomass in the MBBR process volume may grow on the biofilm or in free suspension. However, if the HRT of the process is sufficiently short, then the biomass that may be found in the mixed liquor in suspension is due mostly to detachment of biomass fragments from the biofilm. With shorter HRT, and a significant amount of biomass present as a biofilm, the non-biofilm fraction of the biomass, for which SRT is equal to HRT, is provided no time for significant growth in the reactor. Consequently, this non-biofilm derived fraction of the biomass, or so-called free-living bacteria, will not contribute significantly to the process microbial community nor will the free-living bacterial be significantly represented in the overall inventory of biomass maintained within the process. In the preferred embodiment, the amount of free-living bacteria, or this non-biofilm derived biomass should be minimized in the process ecosystem. The non-biofilm derived biomass will tend to become washed out and not contribute significantly to the process ecosystem given a sufficiently short process HRT.

Given constraints of influent wastewater RBCOD concentration and volumetric flow rate, reducing process HRT means decreasing the process design volume. Decreasing the process design volume means increasing the process volume organic loading rate. A decreased volume is advantageous in the economics of the process capital investment but the process must be able to accommodate high organic loading rates. MBBRs are robust biological treatment systems with ability to work over a wide range of organic loading rates. A highly loaded MBBR is of advantage for both reducing the levels of free-living bacteria and for a competitive design economically. Organic loading rates nominally between 1 and 15 g/L/day are considered to be applicable to methods and process described herein.

In an MBBR process protozoan populations are generally supported to be present in the process as part of the biofilm growth. Protozoa are grazers of bacteria and can effectively reduce the biomass SRT because the average time that any given individual bacterial cell may be present in the process mixed liquor will depend on both the process hydraulic retention time and the probability of predation by protozoa. Since free-living bacteria are often present as individual bacterial cells, protozoan communities in the process may more readily graze upon these individual organisms. Therefore, protozoan communities residing in the biofilm may assist in limiting the contribution of free-living bacteria to the process ecology in addition to designing for and maintaining a sufficiently short process HRT.

Benefits of a moving bed biofilm (MBBR) system include increased resilience to toxicity and variable loading, simple operation, and a biological treatment system that is insensitive to sludge bulking. In an activated sludge based treatment system, all the bioreactor effluent sludge must be continuously separated from the treated water and returned to the treatment basin. This continuous separation of all the biomass is why activated sludge is so sensitive to onset of sludge bulking conditions. In an MBBR process only the excess produced biomass ends up in the effluent stream. The majority of the process biomass is associated with the biocarriers and this active biofilm that is attached to the carriers is detained in the treatment tank by sieves over the outlet(s). With the biocarriers retained in the process volume, only the treated water with just the surplus produced biomass is passed to the effluent and to downstream units for further processing. Retaining the carriers in the main MBBR reactor thereby inherently retains most of the biomass in the process. Therefore, the separation technologies used to remove the surplus biomass from the effluent of an MBBR can be very compact when compared with those used for activated sludge processes. Separability of this biomass is important but the separation is much less sensitive to criteria of sludge volume (bulking). This insensitivity means that MBBRs can work stably with a much wider range of morphologies and species of bacteria. Flexibility to morphologies and species is of added value to the present objectives of enrichment for a PHA accumulating biomass and producing a biomass with enhanced PHA content in one step.

One does not specify a specific growth rate for the biomass in the biofilm for the operation and control of an MBBR process. Furthermore, one does not specify an SRT for the biofilm biomass. Those skilled in the art of MBBR design understand criteria of aeration, mixing, tank volumes, and degree of biocarrier filling towards meeting water quality objectives for treating a wastewater with given organic contamination and temperature. The process is in many respects self-regulating. Surplus biomass is produced as a result of the contaminant removal. MBBRs allow for high volumetric organic loading rates, maintaining effectively more biomass per unit volume and requiring a significantly reduced volumetric foot print than an activated sludge process designed for the same organic contaminant removal. Therefore, we considered a biofilm approach to producing surplus biomass with enhanced PHA content to be of significant practical economic benefit and ease in maintenance and operations.

Those skilled in the art of MBBR design and operation know that the idea behind the development of the MBBR process has been to utilize the best features of the activated sludge process as well as those of the biofilter process without including any of the worst (Ødegaard (2006). Water Science and Technology 53(9):17-33). The MBBR utilises the whole tank volume for biomass growth as for activated sludge, but in contrast to activated sludge, the state of the art for MBBR process design is without any biomass recycle. In common practice no sludge recirculation takes place, only the surplus biomass has to be separated and this is seen as a considerable advantage, with benefits in savings of investment and operating costs, over the activated sludge process. Notwithstanding the well-established conventional wisdom of MBBR design and operation, we found to our surprise that a strategy of selective retention and recycling of effluent MBBR biofilm biomass in combination with MBBR operating conditions of phosphorus limitation with RBCOD/P greater than 200 g/g were conducive to producing a surplus biomass with enhanced PHA content.

MBBR processes used in combination with activated sludge processes, or so-called IFAS (Integrated Fixed-Film Activated Sludge) processes are operated with a sludge recycle of the activated sludge biomass (Di Trapani et al (2013). Biochemical Engineering Journal 77:214-219). MBBR based IFAS processes are generally applied as a means for upgrading and optimising performance of an activated sludge process for improved nitrification, nitrogen removal, and/or nitrogen and phosphorus removal. In distinction to the embodiments of the methods and processes that are presented herein, the sludge recycle in an IFAS process is implemented for maintaining the SRT of the activated sludge in the IFAS process, and it is not directed towards selective retention of MBBR unit process effluent biofilm biomass.

The occurrence of PHA in biomass produced in an MBBR has been reported in the research literature (Pozo et al. (2012). Water Science and Technology, 66(2): 370-376; Pozo et al (2012). Journal of Environmental Science and Health Part A, 47(13):2052-2059), however the operational factors that could be applied to regulate reliable production of a PHA-rich-biomass with enhanced PHA content from an MBBR have, until our findings herein, been poorly understood. The MBBR technology has been a model system for our purpose to understand the methods and process for continuous mixed culture growth with enhanced PHA production because it is a very simple bioprocess to operate.

For an MBBR, a biomass inventory may be maintained predominantly as a biofilm within a completely stirred tank reactor and excess biomass that becomes detached as freely suspended biofilm fragments exits the process in the effluent as volatile suspended solids which may then be separated (harvested) and processed for polymer recovery. We define more specifically the SRT of the MBBR biofilm biomass as SRT_{BB}. SRT_{BB} is generally known to be significantly longer than the process HRT but it is also otherwise generally an uncontrolled process parameter. Even if it is not specifically controlled, the biofilm biomass SRT_{BB} is managed inherently in the process by the rate of growth of the biofilm in combination with the detachment rate of biomass fragments from the biofilm. One embodiment of the methods described herein may be implemented via a process like, for example, the process presented via process schematic in Example 5.

The biofilm biomass generally becomes detached on an ongoing basis, and the detached fragments of biofilm biomass coming from the biofilm generally are present in the mixed liquor in the form of aggregates. Whilst the biomass associated with the biofilm carriers are retained on the biocarriers, and these biocarriers are retained in the process volume of the biofilm process, the detached biofilm aggregates that enter into the mixed liquor will eventually flow freely out of the process volume in the effluent. Without limitation we refer to the process biofilm biomass as being comprised of both biomass attached to the biofilm surfaces, as well as the detached fragments of biofilm found in the mixed liquor as suspended volatile solids.

Since detached biofilm aggregates in the mixed liquor are generally readily separable from the effluent stream, we refer to this fraction of the mixed liquor volatile suspended solids as readily separable mixed liquor volatile suspended solids or RS-MLVSS. The SRT of the biofilm biomass (SRT_{BB}) is thereby composed of two time elements; firstly, the time an element of biofilm biomass spends on average attached within the biofilm (SRT_{BF}) and, secondly, the time on average the detached aggregates of RS-MLVSS biomass spend in the mixed liquor once they become detached from the biofilm (SRT_{RS}):

SRT_{BB}=SRT_{BF}+SRT_{RS}

In conventional MBBR operation, the SRT_{BB} and the SRT_{BF} are generally uncontrolled and unknown. Further, the SRT_{RS} is generally uncontrolled and known to be inherently equal to the bioreactor HRT in the established conventional MBBR design and operation methods. We have found that, in contrast to such common practice, by explicitly taking control of the SRT_{RS} and controlling the SRT_{RS} to be greater than the bioreactor HRT, it is possible to produce a surplus biomass from a biofilm process with enhanced PHA content.

The design SRT for activated sludge in a high rate activated sludge process for carbon removal is typically between 3 and 10 days. A sufficiently long SRT is required in activated sludge to maintain an optimal food to micro-organism ratio range with respect to the activated sludge biomass in order to maintain the treatment performance in carbon removal alongside an activated sludge with good settling properties for sludge recycling. The SRT_{RS} control is not to be confused with SRT control for an activated sludge. SRT_{RS} control is not directed towards maintaining and controlling an activated sludge within an MBBR process. SRT_{RS} control is directed towards applying our discovery of an influence from reducing the specific organic loading rate to the biofilm biomass in order to stimulate an enhanced PHA content of the biomass comprising the effluent MLVSS. In the preferred embodiment SRT_{RS} should not significantly exceed the MBBR HRT and preferably it should be less than 1 day.

By selectively controlling the SRT_{RS} one effectively can modulate and control the inventory of biofilm biomass in the biofilm process. We have found that decreasing the specific organic loading rate to the biofilm biomass promotes a greater expression of the PHA accumulating potential from the biofilm biomass under conditions of operation with RBCOD/P greater than 200 g/g. Control of the biofilm biomass specific organic loading rate can be achieved by increasing the available surface area for biofilm growth (more carriers in the reactor). Notwithstanding, we find that the effect of biofilm specific organic loading rate can be further augmented and more directly and actively tuned and controlled by selective retention of the RS-MLVSS so as to increase the SRT_{RS} to be greater than the process HRT.

In the illustrative process depicted in Example 5, it is preferable to produce the biomass as aggregates. In activated sludge processes, aggregates may be suspended flocs or granules of biomass that may readily be separated from the effluent using sedimentation or flotation unit processes. In biofilm systems, the aggregates are the fragments of excess biomass that grow out and become detached from the biofilm. Fragments of biomass may also be released due to shear forces that dislodge biomass from the surface. Such biofilm fragments, namely the RS-MLVSS, are also readily separated from the bioprocess effluent based on well-established separation methods that are generally known to experts in the field and may include unit separation processes such as flotation and ballasted sedimentation. However, based on the current state-of-the-art, a fraction of the effluent separated RS-MLVSS is not generally selectively recycled back to the MBBR unit process.

The separation of the RS-MLVSS is considered to be selective if the methods of separation do not tend to also retain and recycle non RS-MLVSS. By non RS-MLVSS we refer to so-called free-living bacteria that are not generally present as aggregates of biomass. Thus while RS-MLVSS is made to have a SRT_{RS} that is greater than the process HRT, in the preferred embodiment, the free-living (FL) bacteria that comprise a fraction of the effluent MLVSS will be nevertheless maintained with an SRT that is still equal to or less than the process HRT (SRT_{FL}≦HRT).

In biofilm processes, the aggregates of detached biofilm may occur passively due to the biofilm growth. Detachment may also be accomplished actively by periodically imposing mechanical or hydraulic forces on to at least some biocarrier elements carrying attached biofilm at any given time. The SRT_{RS} can be controlled to be longer than the process HRT by separating at least a fraction of the RS-MLVSS in the process effluent, and returning at least a fraction of this separated fraction back to the MBBR process volume.

In a preferred embodiment, the process operating conditions should be such that free-living bacteria do not form a dominant fraction of the process biomass. Free-living bacteria are motile or non-motile, and generally comprise single bacterial cells that may tend to proliferate in a bioprocess given a sufficient combination of time (HRT), temperature and availability of RBCOD as well as other nutrients that are necessary for growth. The SRT with respect to the free-living bacteria is approximately equal to the HRT since they are more difficult to separate and retain within a continuous flow through bioprocess without, for example, membrane separation. If protozoan grazing activity is significant, then the SRT for the free-living bacteria (or SRT_{FL}) is even less than the process HRT. Generally, methods that may be applied to separate RS-MLVSS from the process effluent are not effective to separate free-living bacteria from the effluent. In the preferred embodiment, the objective is to increase SRT_{RS} to be greater than the process HRT, while maintaining SRT_{FL} to remain equal to or less than the process HRT. Therefore in the preferred embodiment, a separation method for the preferential separation of the RS-MLVSS is to be applied wherein fee-living bacteria are not captured and concentrated by the separation methods. It is a process objective to minimize the contribution of free-living bacteria MLVSS (FL-MLVSS) to the process MLVSS:

MLVSS=FL-MLVSS+RS-MLVSS, where FL-MLVSS<<RS-MLVSS
such that ideally, MLVSS=RS-MLVSS

The higher the (mesophilic) temperature, the shorter the HRT needs to be, for example in an MBBR process, in order to establish domination of biofilm growth with only minor presence of biomass as free-living bacteria. In a preferred embodiment, an MBBR is operated with an HRT of less than 3 hours within a temperature range from 20 to 55° C. such that more than 50% of the influent RBCOD is removed within the bioprocess HRT. A sufficiently short HRT is selected such that more than 55%, preferably more than 75% and most preferably more than 95% of the effluent biomass suspended solids are not comprised of free-living bacteria, but rather may be considered to be aggregates that are most probably due to detachment and washout of biofilm fragments:

RS-MLVSS/MLVSS>0.55

We consider that aggregates of biomass are readily separable due to reasons of aggregate size and/or morphology that permit for effective separation methods of the aggregated biomass. Such methods may apply principles, for example, of density difference (gravity settling or floatation), and or particle size exclusion (micro-screening). Whereas biomass in the form of suspended aggregates in mixed liquor are amenable to one or more of these principles of separation, non-aggregated free-living bacteria are not. Therefore, the MBBR mixed liquor volatile suspended solids (MLVSS) that are aggregated and thereby readily separable are considered to have origin from the biofilm. Herein, further reference to the readily separable fraction of the MLVSS refers generally to the fraction of biomass present as aggregates in the process mixed liquor, and aggregates of biomass that have interpreted origin due to the processes of biofilm growth and biomass detachment from the biofilm.

Discrimination and quantification of MLVSS biomass fractions present as free-living or present as aggregates (defined operationally as readily separable) may be determined by diagnostic microscopy for instance. In combination with microscopy, other quantitative methods for measuring suspended solids can be used to characterize the biomass compositional fractions and, in so doing, an optimally low HRT may be determined. For example, sample centrifugation (sedimentation) with selected g-force and centrifugation (sedimentation) times may be used to segregate biofilm aggregates from free-living bacterial fractions. Alternatively filtration methods using selected filter pore sizes from 0.45 to 1.6 and to 3 μm may be used in a similar fashion to segregate and quantify the constituent biomass fractions and, in so doing, determine the most suitable HRT for a minimal desired low level of free-living bacterial biomass in the process effluent MLVSS. HRT is a function of influent volumetric flow rate and bioprocess volume, thus HRT may be adjusted by either of these process parameters.

In general it is of disadvantage to produce free-living biomass from a water treatment process. This disadvantage is further underlined economically if a process objective is to maximize the yield in production of a readily separable value-added biomass with enhanced PHA content. Free-living biomass in the effluent of a biofilm process is generally difficult to separate. Even if this free-living biomass were to contain PHA, it may be economically prohibitive to establish industrial methods to separate this kind of non-aggregated biomass from the effluent stream of a process water or wastewater treatment system. Thus one objective of the methods discussed herein has been to establish maximum production of PHA-rich-biomass from the treatment of RBCOD from influent water, wherein the produced biomass may be readily separated from the effluent in order to maximize overall industrial economic gains in conversion of organic contamination in a wastewater to a value added polymer as a renewable resource.

HRT determination for a given influent RBCOD and operating temperature is complicated but can generally be established empirically. One way that HRT may be determined is using laboratory testing. For example (Piculell et al. 2014, Water Science and Technology. 69(1), 55-61), tests were performed where RBCOD is in the form of acetic acid and where HRTs of 1.2, 2.4 and 3.4 hours were tested for a process operated at 10° C. In these experiments the contributions of biofilm biomass to the RBCOD removal were examined in short term tests where carrier material was removed from the process volume. Here it was shown that RBCOD removal without carriers was negligible at an HRT of 1.2 hours, and became measureable with HRTs of 2.4 and 3.4 hours. In another example, an influence of HRT in an MBBR process operated at 20° C. treating an influent water with glucose and acetate as RBCOD was demonstrated. The effluent suspended solids were measured in a well-mixed sample and in the sample supernatant after allowing for 30 minutes of settling (Karizmeh et al. 2014, Bioprocess and Biosystems Engineering, 09/2014, 37(9):1839-1848). These disclosed methods may be applied operationally towards establishing process parameters in design as well as for the process monitoring routines as described and applied in Example 7.

Since free-living growth in the effluent from a biofilm process does not generally settle very well, settling provides for a rough discrimination of biofilm derived biomass from free-living biomass. In one literature example (Karizmeh et al. 2014, Bioprocess and Biosystems Engineering, 09/2014, 37(9):1839-1848), for a given organic loading rate to the process, the suspended solids concentration in the effluent supernatant was observed to decrease exponentially with HRTs from 2 hours down to 1 hour. The fraction of non-settleable suspended solids also decreased progressively from with HRT of 2 hours down to 1 hour. Similarly we established based on simple field methods of COD measurement and a 2-hour settling test reference time, a dominant fraction of RS-MLVSS in the effluent of an MBBR process (Example 7) producing a PHA-rich biomass with a 1-hour HRT.

The above-mentioned examples illustrate, without limitation, that methods exist to readily evaluate an influence of HRT on the interpreted production of biofilm and non-biofilm biomass from an MBBR process. Further, even at process operating temperatures at 20° C. and below, HRTs of less than 1-hour have been shown to be preferable in the literature towards reducing contribution of RBCOD removal by free-living biomass in an MBBR process. A significant (exponential) increase of non-biofilm biomass has been indicated for RBCOD removal with increasing HRT up to 2 hours.

For many species of bacteria typical doubling (reproduction) times are in the order of 20 to 30 minutes. Generally, reproduction rates in the mesophilic temperature range (20 to 45° C.) are observed to increase with temperature with a maximum reproduction rate around 37° C. This suggests that for higher temperatures a washout HRT for the free-living biomass may need to be theoretically lower than 0.5 hours. Notwithstanding, due to the presence of carriers that may support protozoan biomass in the biofilm growth and the disclosed methods of disposing the biomass to a phosphorus limitation, longer HRTs may be possible than estimated based on theoretical growth rates. A combination of a sufficiently low HRT, some form of growth limitation, and predation by protozoa residing with the biofilm create conditions to maintain a low contribution of RBCOD removal by the free-living biomass.

A wastewater may contain COD where only a fraction of the COD is present as RBCOD. The methods described herein relate to continuous (or semi-continuous) processes for the production of a PHA-rich-biomass in a single step of phenotype selection during biomass growth on RBCOD with concurrent PHA production. The HRT is to be sufficiently long, to remove more than 50%, preferably more than 70% and even more preferably more than 90% of the influent RBCOD, while still being sufficiently short to avoid unwanted excess growth of free-living bacteria. The RBCOD is to be used most preferably by the selected populations of bacteria as a carbon source for combined active biomass growth and PHA storage metabolic activities. Generally, the non-RBCOD fraction of the influent COD may pass through the PHA production process and become treated subsequently if needed in further downstream unit process steps of water treatment (Example 5).

In the first instance the purpose of the water treatment process described herein relates to the efficient and robust removal of RBCOD from a wastewater. We have found that by appropriate selection of HRT, organic loading rate, and nutrient balance to the influent RBCOD, the process will enrich for the PHA-storing phenotype in the biomass and produce a surplus biomass with enhanced PHA content. One preferred embodiment is for the continuous production of a PHA-rich-biomass as a result of the treatment of RBCOD from a wastewater. Just the same, given that a primary demand on the process may be water quality improvement and not only PHA production, and without limitation, the conditions for PHA production may not be readily applied continuously; therefore the process may be utilized in periods or campaigns for PHA production. Another embodiment is directed towards non-continuous production of PHA in surplus biomass from the treatment of RBCOD from a wastewater.

The present disclosure further describes a method for enrichment of a biomass with the PHA-storing phenotype and maintaining process-operating conditions that result in enhanced biomass PHA content of more than 30%, and preferably more than 40% (gPHA/gVSS). We have found that phosphorus limitation can be applied to selectively enrich a biomass with the phenotype of PHA producers; further an elevated enhanced level of the PHA content in the surplus biomass is sustained by maintaining the biomass with capacity for higher maximum extant respiration rate wherein the specific organic loading rate is reduced as much as possible. The specific organic loading rate is the process mass input rate of RBCOD (kgRBCOD/d) divided by the process biomass content (kg-biomass).

The biomass was observed be sustained to maintain a higher capacity for rate in COD removal by repeatedly exposing at least a fraction of the biomass at any given time to a zone of elevated RBCOD concentration. The biomass expression of PAP was found to be increased by such repeated stimulation and the effluent biomass PHA content was found to be enhanced by reducing the process specific organic loading rate. The specific organic loading rate was reduced by providing for an increase in the amount of biomass in the process. In an MBBR process, a potential for a greater amount of biomass may be provided for by increasing the number of carrier elements (to increase the available surface area for biofilm development) present in the process and/or by selectively increasing the SRT_{RS} to be greater than the process HRT.

Notwithstanding, the strategy to provide for more biomass in the process by increasing the number of biocarrier elements in mixed liquor, the biomass in the process may also be increased by the selective retention of the readily separable MLVSS (RS-MLVSS) as described above. Applying methods to selectively retain effluent RS-MLVSS in the process volume are a means to selectively increase the SRT_{RS} of the RS-MLVSS in the process. Selective retention of the RS-MLVSS is furthermore a means to more actively control the increase in the biomass in the process due to an increase the concentration RS-MLVSS in the process mixed liquor as a result of controlling SRT_{RS} to be greater than the process HRT.

The production of a PHA-rich-biomass was found to be applicable over a wide range of operating temperatures. Temperature is an important process parameter related to both control of process ecology as well as operating costs in biological treatment of industrial process waters and wastewaters. Generally it is expensive to heat water up and, conversely, some industries prefer that water quality of internal process waters may be managed without the need and undue expense to cool the process waters down. We have found that the methods and process are generally applicable to bioprocess operating temperatures that are between 20° C. and 60° C. In experiments under thermophilic operating conditions, PHA production with PHA content above 40% (g-PHA/g-VSS) was achieved. We have observed in some cases that temperatures that are lower (22° C.) may tend to select also for a fungal biomass. Therefore, we find that in at least some cases it is preferable to maintain average process temperatures greater than 25° C. and less than 55° C. As optimal temperature is dependent on many factors, in some embodiments, laboratory tests will be necessary to determine optimal temperatures prior to utilizing the methods described herein. Examples 1, 2, and 3 are illustrative of such laboratory testing methods.

Also, the disclosed principles of single step PHA production have been found to be applicable over a wide range of supply (loading) rate of nitrogen (N) with respect to the RBCOD supply rate. Relative N-loading rates in excess of those considered to be necessary for the stoichiometry required for biomass growth may be applied. However, supply of nitrogen in excess of the process requirements may add unnecessary operating expense of chemical addition. Therefore, it is of interest to approach conditions maintaining a minimal required N-loading to the process where nitrogen is otherwise limiting in the wastewater. However, we found that when N-loading conditions became insufficient, the RBCOD removal performance of the process suffered. Therefore, in the preferred embodiment, N-loading rates should be sufficient to ensure greater than 50, preferably more than 70, and most preferably more than 90 percent removal of the influent RBCOD.

The amount of N to be supplied with influent RBCOD may be specified as a RBCOD/N ratio. The nitrogen supplied and specified in this ratio is to be present in a form that is readily bioavailable and can thereby be readily assimilated by the biomass in combination with RBCOD to generally satisfy metabolic requirements for biomass maintenance and growth. The RBCOD/N supplied may be facilitated through the combination of multiple RBCOD and N containing streams, as well as separate chemical additions. A preferred embodiment is a single step biofilm PHA production process where influent RBCOD/N is maintained on average between 20 and 70 g/g, and more preferably on average between 30 and 60 g/g.

The principal selection pressure for the enrichment of the PHA storing phenotype is by means of maintaining control of the amount of phosphorus supplied in relation to the amount of RBCOD supplied to the process. We have also found that it is preferable if the RBCOD is dominated by volatile fatty acids (VFAs). VFAs are known platform chemicals that may be converted by many species of bacteria into PHAs. Notwithstanding the fact that other forms of RBCOD may also be converted into PHA, our testing to date suggests that it is preferable that at least 50% and more preferably greater than 70% of the RBCOD in the influent to the process be present (on a COD basis) as VFAs. Where the VFA content of the influent COD is lower, fermentation pre-treatment may be applied to increase RBCOD content of the COD and the VFA content of the RBCOD prior to the PHA production step (Example 5).

The methods and processes disclosed herein are well adapted to embodiments for the treatment of nutrient limited process waters and wastewaters with the principal objective to improve water quality by reducing RBCOD concentration. Biological treatment systems are effective to reduce RBCOD from process waters and wastewaters but generally biological treatment systems utilize a specific balance of COD, nitrogen, and phosphorus as well as other trace nutrients that are necessary to support biomass metabolic activity in general. Many industrial process waters from, for example, pulp and paper industries as well as agricultural processing, and food and beverage industries, to name just a few, may be deficient in the amounts of nitrogen, phosphorus and/or other trace nutrients, such as certain metals, for the stable and reliable biological removal of soluble COD. Therefore, nutrients may be added for a biological treatment process and the cost of nutrients may represent a significant fraction of the treatment operating expenses. We found that, in combination with SRT_{RS} control, restricting nutrient addition, and more specifically phosphorus, is a principal factor for increasing selection of a biomass with PHA accumulating potential. Therefore, the methods described herein are intended to bring added value to the investments of performing essential services of water quality management, as well as contributing to reduced cost of operations due to less nutrient demands.

A PHA storage response may be triggered by the ability of a biomass to redistribute phosphorus (P) easier than nitrogen under conditions of P starvation. P-limitation has been considered to promote conditions where more organic carbon becomes redirected towards PHA synthesis given that the Krebs cycles is restrained due to limited activity of ATP synthase (Cavaillé, et al. (2013). Bioresource Technology, 149:301-309). In this way, a sustained pressure in a continuous flow system through P-limitation would tend to encourage a flux of carbon towards PHA synthesis. While some process waters and wastewaters may be with nutrient concentrations in excess of heterotrophic bacterial growth requirements, many others are rich in RBCOD but relatively poor in nutrients making it necessary to add N and/or P in order to operate a biological treatment process for the purpose of COD removal. P-limitation may be created by supplemental addition of phosphorus to a P-deficient wastewater, or else supplemental RBCOD addition to a P-sufficient wastewater. Additions to achieve desired RBCOD/N and RBCOD/P influent values may be made via, for example, N-streams, P-streams, RBCOD streams and/or streams maintaining a mix in combinations of RBCOD, N and P.

The amount of P to be supplied with influent RBCOD may be specified as a RBCOD/P ratio. The phosphorus supplied and specified in this ratio is present in a form that is readily bioavailable and can thereby be readily assimilated by the biomass in combination with RBCOD to generally satisfy metabolic requirements for biomass maintenance and growth. The RBCOD/P supplied may be facilitated through the combination of multiple RBCOD and P containing streams, as well as separate chemical additions. A preferred embodiment is a single step biofilm PHA production process where influent RBCOD/P is maintained on average between 200 and 800 g/g, and more preferably on average between 300 and 800 g/g so as to maintain an effluent mixed liquor containing biomass volatile suspended solids (VSS) with PHA content exceeding on average 30% gPHA/gVSS.

We find that conditions, of relatively high organic loading rate (OLR>2 gCOD/m³/d) with a RBCOD dominate feedstock, under phosphorus balanced or excess loading conditions (RBCOD/P≦200 g/g) are conducive to producing a biomass that is enriched with the PHA storing phenotype. The problem we found was that the PHA accumulation potential of the biomass is not always expressed adequately in the effluent suspended solids. Stepwise decreasing of RBCOD/P from balanced to limiting conditions (RBCOD/P≧200 g/g) resulted in progressive increase in the MLVSS PHA content to levels that are greater than 30% gPHA/gVSS. Further we found that increasing the process biomass content by means of selective retention of at least a fraction of the effluent RS-MLVSS, promoted for preferred conditions of an effluent MLVSS with enhanced PHA content greater than 40% gPHA/gVSS.

We consider that more stable operating conditions are achieved by means of imposing phosphorus-limiting conditions by steps in gradual shift from phosphorus balanced to phosphorus limiting conditions. As P-limiting conditions are applied, in order to sustain the process kinetics of RBCOD removal, more active biomass is also required in the process. More active biomass can be maintained in an MBBR process by increasing the available surface area for biofilm growth, or by a progressive implementation of selective retention of at least a fraction of the effluent RS-MLVSS. SRT_{RS} can be increased in takt with step increases of RBCOD/P so as to main an active control of the specific organic loading rate.

We confirmed the strategy of an industrial process using P-limitation as an environmental pressure to enhance selection for the PHA-storing phenotype in a biofilm biological treatment process. Nutrient limitation, and the significant removal of influent RBCOD within the HRT of the process step ensures, in addition to P-limitation, a scarcity of excess RBCOD that may allow in general for non-PHA-storing phenotypic bacteria to grow and proliferate in the system. The combination of P-limitation in COD removal and a bioprocess environment that creates for RBCOD limitation were observed to be sufficient conditions for the enrichment of the biomass with the PHA-storing phenotype. However, these bioreactor environmental conditions of combined P and RBCOD limitation were, to our surprise, not found to be sufficient to sustain a stable enhanced PHA level in the surplus biomass. We discovered that while enrichment of the biomass was accomplished due to the RBCOD under P-balanced and P-limitation, an enhanced PHA content of the produced biomass was achieved in an MBBR process in association to an increased process biomass content while progressively applying P-limitation. Effective decrease in the process specific organic loading rate, either by maintaining more biofilm bio-volume, or by selective retention of RS-MLVSS created for conditions of enhanced effluent biomass PHA content.

The increased process biomass content is synonymous to a decrease in the process specific organic loading rate, and this parameter may be readily controlled, for example, by increasing the SRT_{RS} of the RS-MLVSS. SRT_{RS} of the RS-MLVSS is increased by the selective retention of at least a fraction of the effluent RS-MLVSS. Example 8 illustrates embodiments of RS-MLVSS selective retention without limitation of other means to establish similar principles by those skilled in the art of biomass separation and bioreactor technologies and their design. The SRT_{RS} of the RS-MLVSS may be defined by the mass of RS-MLVSS in the process volume divided by the mass of RS-MLVSS removed from the process per day.

An enhanced PHA content was observed when the specific organic loading to the process was reduced. The specific organic loading is the rate of RBCOD supplied with respect to the total biomass in the process. When less RBCOD is available per unit of biomass, one may interpret that kinetics of PHA storage become more advantageous for survival than kinetics of biomass growth. Bacteria that are adapted to sequester the organic carbon and assimilate PHA more quickly are more competitive because they get and control a limited resource (RBCOD) as an intermediary intracellular storage product (PHA). Notwithstanding the possibility for future insights to the process, we found that an enhanced PHA content in the surplus biomass became sustained when the available surface area for biofilm development was increased while keeping the process organic loading rate and HRT otherwise constant. Similar results were also obtained by selective retention of the RS-MLVSS while keeping the available biofilm surface area constant. With increased process biofilm surface area, the biofilm biomass as observed to increase and as the biomass content of the MBBR increased, the specific loading at constant organic loading rate decreased, and this was correlated to enhanced PHA content in the surplus biomass. Similarly, we found that when selective retention of RS-MLVSS was applied under conditions of P-limitation, PHA content in the effluent suspended solids became enhanced to preferred levels of greater than 40% gPHA/gVSS.

For a biofilm process like an MBBR, we define a biofilm bio-volume whereby the bio-volume is in the first instance defined by the so-called protected surface area where biofilm may develop with minimal risk for mechanical contact with other surfaces when contact and collisions take place between walls, other MBBR elements, and any other internal structures in the reactor. The protected surface area is therefore created by virtue of the geometry and features of the MBBR element design in relation to both the reactor into which they are placed as well as to each other. For example, Example 6 illustrates an MBBR biofilm carrier element whereby the protected surface areas are defined by a matrix of rectangular wells. The biofilm that builds within respective wells is protected to a great extent from surfaces and protrusions that are larger in area than the well size. In Example 6, the well size is approximately 2.3×2.3 mm and each carrier is with 208 (104 per side) such wells. Biofilm that grows outside of the confines of the well will with high probability be detached due to contact with other carrier elements. The depth of the biofilm further defines the biofilm bio-volume, where it is generally observed that beyond a biofilm thickness of 0.200 mm, oxygen penetration into the biofilm becomes negligible. Therefore the process maximum aerobic biofilm bio-volume per MBBR biocarrier element may be estimated for the Z-MBBR carrier as 2.3×2.3×0.2 mm×208, or 220 mm³ or 0.22 mL. For an MBBR process with 200,000 elements per m³, a process maximum biofilm bio-volume may be estimated to be about 44 L. Based on this calculation, those skilled in the art will understand methods to increase the available amount of the process biofilm bio-volume as a strategy to decrease the process specific organic loading rate, with an objective to produce an excess biomass with enhanced PHA content.

Thus one example of an appropriate biocarrier is the Z-MBBR biocarrier discussed in Example 6. An advantage of the Z-MBBR biocarrier (or other designs of an analogous nature) is that the projected biocarrier biofilm surface area does not change with biofilm growth on the surface, and the biofilm formation is protected as a function of the well depth. Z-MBBR biocarriers with different well depths can be made for which the well depth is preferably less than 0.4 mm, more preferably less than 0.3 mm and most preferably less than 0.2 mm. Therefore the process maximum biofilm bio-volume can be readily estimated as illustrated above and an optimum biomass PHA content may be established empirically by selecting a sufficiently large maximum biofilm bio-volume for removing most of the influent RBCOD with a given organic loading rate, temperature and HRT.

Other examples of appropriate biocarriers that allow for maximizing bio-volume include the use of sand particles as the biofilm carrier elements (Nicolella et al. (2000), Journal of Biotechnology, 80(1):1-33). Sand particles can be fluidized by up-flow or with the benefit of an airlift reactor design. Biofilm growth is nested and protected with a base in the sand crevices much like for the Z-MBBR wells. The biofilm grows out and a roughly spherical biofilm forms with the process biofilm bio-volume now becoming dependent on the number of particles added to the process and the average particle-biofilm radius that is maintained in the process. Knowledge of the average particle size and measurements of the average size of the particle-biofilm combination in the process can be used to assess the extant process biofilm bio-volume during operations. Process operating conditions like mixing intensity causing shear stress to the biofilm will limit the extent to which the biofilm may grow radially from the particle. Effluent flow of biomass as detached biofilm or larger particle-biofilm elements that may be carried out of the fluidized bed may be harvested and/or selectively recycled in part. The process biofilm bio-volume can be maintained with fewer particles and thicker biofilms or a greater density of particles per unit volume with a thinner maintained biofilm.

Operational control of biofilm bio-volume may not always be practically feasible. However, greater than a sufficient biofilm bio-volume should be provided in the process. A sufficient biofilm bio-volume is determined by the amount of biofilm bio-volume specified wherein further increase in available biofilm bio-volume does not result in increased rates of RBCOD removal for given operating conditions specified by RBCOD loading rate, HRT and temperature. Sufficient biofilm bio-volume is to be determined under operating conditions without nutrient limitation and where SRT_{RS} is equal to HRT.

Without limitation, the biofilm biomass may be harvested (detached) passively or actively. Passive harvesting relies on the on-going detachment of biofilm from the biofilm carriers. Since the carriers are generally retained in the process, suspended biomass in the effluent mixed liquor is separated to produce a concentrated stream of suspended solids and final effluent discharge stream of relatively low suspended solids. Active harvesting from the biofilm relies on the periodically exposing at least some of the carrier elements in the MBBR process to mechanical forces or else elevated hydraulic mixing intensities in order to generate shear stresses that are higher than would normally be experienced in the main MBBR. Under these conditions of periodically applied elevated shear stresses, a greater amount of biofilm may be detached, separated, and concentrated.

In general we found that the PHA content of the biomass became enhanced when most but not all of the bio-volume capacity of an MBBR process is utilized. In particular, an increase in available bio-volume correlates with an increase in the amount of biofilm and an increase in RBCOD removal. By increasing the amount of biofilm biomass present in the process, PHA storage by the biomass is enhanced. By increasing the available maximum biofilm bio-volume in the process one establishes conditions to minimize the specific loading organic loading rate. We have found that minimizing the specific organic loading rate while operating an MBBR under conditions of P-limitation provides for the production of a biomass with enhanced PHA-content. Specific organic loading rate can similarly be modulated for a given available process biofilm bio-volume by controlling SRT_{RS} to be greater than HRT.

Based on findings presented in Example 8 of a nominal 10% variability in MLVSS concentration while operating an MBBR with SRT_{RS} equal to HRT, we consider that conditions of SRT_{RS} greater than HRT are met when a sufficiently elevated average MLVSS concentration is sustained in the biofilm process volume mixed liquor. Thus based on the findings of Example 8, recycling a sufficient amount of RS-MLVSS to maintain an average concentration of RS-MLVSS in the biofilm process that is at least 10% higher than an average RS-MLVSS concentration for the same biofilm process and operating conditions but without any RS-MLVSS recycling establishes conditions of SRT_{RS} to be greater than HRT.

The amount of biofilm bio-volume capacity to be present in a process depends on organic loading rate. The higher the organic loading rate the greater the maximum available biofilm bio-volume in the process must be in order to reach the same lowered specific organic loading rate. In the case of an MBBR, more bio-volume may be supplied by increasing the number of carrier elements in the process all the way up to such a point that other factors become of practical limitation, such as the feasible filling degree of carriers or the feasible supply rate of oxygen (or nitrate) to the process. One may, in an analogous fashion, decrease the specific loading rate by selectively increasing the SRT_{RS} of the RS-MLVSS to be greater than the process HRT, and in so doing it is possible to reduce the process specific organic loading rate by increasing the process biomass content with respect to the RS-MLVSS. Since the RS-MLVSS is interpreted to be dominated by biomass of biofilm origin, controlling the RS-MLVSS SRT is a means to achieve the same desired outcome from supplying more biofilm bio-volume to the process. We believe that SRT_{RS} control provides for a robust method to establish, on average, conditions for enhanced PHA content as the inventory of biomass in the biofilm may fluctuate periodically due to otherwise uncontrolled events of biofilm detachment.

A sustained total biomass activity within a mixed liquor that is otherwise of reduced RBCOD concentration was found to support sustained PHA production. We observed that when the RBCOD removal efficiency of the process decreased, whereby the mixed liquor was with increased concentrations of untreated RBCOD, the PHA content of the biomass decreased significantly.

The organic loading rate, with respect to supplied RBCOD per unit volume of reactor, drives the respiration response of the biomass. One method to maintain a biomass, in a mixed liquor of relatively low RBCOD concentration, with maximal or near maximal respiration rate capacity is by periodically and repeatedly exposing and stimulating at least part of the process biomass at any given time to an elevated RBCOD concentration.

One way to accomplish this repeated and periodic exposure and stimulation is to use a reactor with at least two zones. One zone, a stimulation zone, stimulates the biomass to higher respiration rate. Zones of biomass stimulation may be created by repeatedly bringing the parts of the process biomass into contact with elevated levels of influent RBCOD. A maximal respiration response may be generated in a biomass by exposure to RBCOD concentrations in excess of 100 mg/L, and preferably less than 2000 mg/L. A second zone, or maintenance zone, is used to hold the biomass while the biomass is not in the stimulation zone. Biomass in the stimulation zone is circulated to biomass in the maintenance zone, and biomass in the maintenance zone is circulated to the stimulation zone. The circulation of the biomass is done periodically such that the respiration rate in the maintenance zone, where RBCOD concentrations are reduced, is maintained as close as possible to the extant maximum biomass respiration rate. In some embodiments, the respiration rate may be maintained in the maintenance zone in mixed liquor with RBCOD concentrations of less than 50 mg/L, but most preferably less than 25 mg/L.

The volumetric capacity for RBCOD removal will depend on the amount of active biomass that can be maintained in the reactor and the ability to maintain that biomass at respiration rates close to the extant maximum. One way to increase biomass respiration rates so that they are maintained close to the extant maximum is by increasing the frequency of exposure of the biomass to the stimulation zones. One way to regulate the amount of active biomass that can be maintained in the reactor is by controlling the available bio-volume, and/or by the selective retention of at least a fraction of the RS-MLVSS such that the SRT_{RS} of the RS-MLVSS is greater than the process HRT. We have found that repeated stimulation of the process biomass improves the biomass expression of extant PHA accumulation potential, and maintains a process biomass with higher capacity for rate of COD removal, with all other factors being equal.

The active biomass may be estimated by the total biomass less the mass of PHA in this biomass. The greater the organic loading rate of RBCOD to the process, the more active biomass the process requires per unit volume. In an MBBR process, the available biofilm bio-volume defines a limit, for the process volumetric carrying capacity for biomass, given a sufficiently short HRT that minimizes proliferation of rapidly growing free-living bacterial biomass. A selected range of P-limitation has been found to enrich for a biomass with the PHA storing phenotype. Increased PHA content of the biomass produced, from a one-step process with at least periods of continuous biomass growth along side significant PHA storage metabolic activity, has been found to occur by providing for a process capacity in biofilm bio-volume that achieves a greater extent of RBCOD removal. As the active biomass grows in the process and the specific organic loading rate decreases, while still maintaining high respiration rate and removing more of the influent RBCOD, the PHA content of the surplus biomass was observed to become enhanced. Under these conditions the process may be maintained for at least periods or campaigns as a continuous 1-step biofilm PHA production process.

Additional support for the claims is available from the findings from experiments, which are disclosed in examples herein. In these experiments we considered process-operating conditions from which we have arrived at embodiments of preferred ranges in phosphorus limitation, ranges in relative nitrogen loading rate, influence of temperature, and carbon limitation, and the methods and process to achieve enhanced PHA content in a surplus biomass produced from the biofilm biological treatment of a wastewater.

### Example 1: Evaluation of RBCOD/P and RBCOD/N on Biofilm Biomass Production and RBCOD Removal

Methods and Materials.

MBBR processes were operated in parallel at laboratory scale. Each process consisted of an aerated reactor with nominally 0.4 L working volume. The reactors were inoculated with biomass as activated sludge collected from the Lund municipal VWVTP, Sweden. Additions of bio-available forms of phosphorus (P) as KH₂PO₄ and nitrogen (N) as NH₄Cl to the influent flow were made to adjust to selected influent organic carbon to bioavailable nutrient ratios of RBCOD/N and RBCOD/P. Trace elements were also added in order to ensure that micronutrients for the biological process were not limiting. The composition of the trace nutrient solution and additions are described elsewhere (Bengtsson, 2009, Biotechnology and Bioengineering, 104(4):698-708).

The excess biomass produced in the process was separated from the effluent and composite samples collected over 1 HRT were used for quantitative assessment of the biomass PHA content. PHA content was determined by thermogravimetric analysis of dried biomass samples (see US 2013/0203954 A1). The fate of COD, nitrogen and phosphorus across the MBBRs were monitored. Conversion and removal rates were estimated based on mass balance considerations.

Two reactors were operated in parallel with a constant organic loading rate of 4 g-RBCOD/L/d and an HRT of 1.5 hours. Temperature was initially controlled at 22° C. and later in the study it was increased to 37° C. A volumetric filling degree of 40% was applied with AnoxKaldnes K5 carriers. One MBBR was an experimental control and in this reference process the RBCOD was acetic acid. The second MBBR was fed with influent RBCOD in the form of a fermented recycle paper mill process water whereby fermentation provided for an increase the VFA content from about 20 to about 91 percent gCOD/gRBCOD. The RBCOD content of the fermented paper mill process water was conservatively estimated based on a respirometric test: SS-EN ISO 1899:1998. These test results were benchmarked against daily routine effluent water quality monitoring and verified through statistical analyses of historical trends.

The reactors were continuously operated over a period of 360 days and during this operation treatment performance and surplus biomass PHA content were assessed. Selected ratios of RBCOD/P from 200 to 1100 g/g with a constant RBCOD/N of 30 g/g were applied in campaigns to assess the influence of P-limitation. Similarly, under conditions of P-limitation meeting the most preferred RBCOD/P embodiment, selected ratios of RBCOD/N were applied in the range of 100≧RBCOD/N≧20 g/g.

Results and Discussion.

Nile red staining of the bacteria was used initially and indicated for enrichment of the PHA storing phenotype in the biomass with positive results. Notwithstanding, with operating conditions in the low mesophilic range (22° C.) we also observed a competition to establish an enriched PHA storing biomass due to growth of microfungi. Bacteria and fungi may compete for nutrients in the environment and both may produce inhibitory products towards the other. By increasing the process temperature to be greater than 22° C. we found that the presence of microfungi became negligible in the biomass.

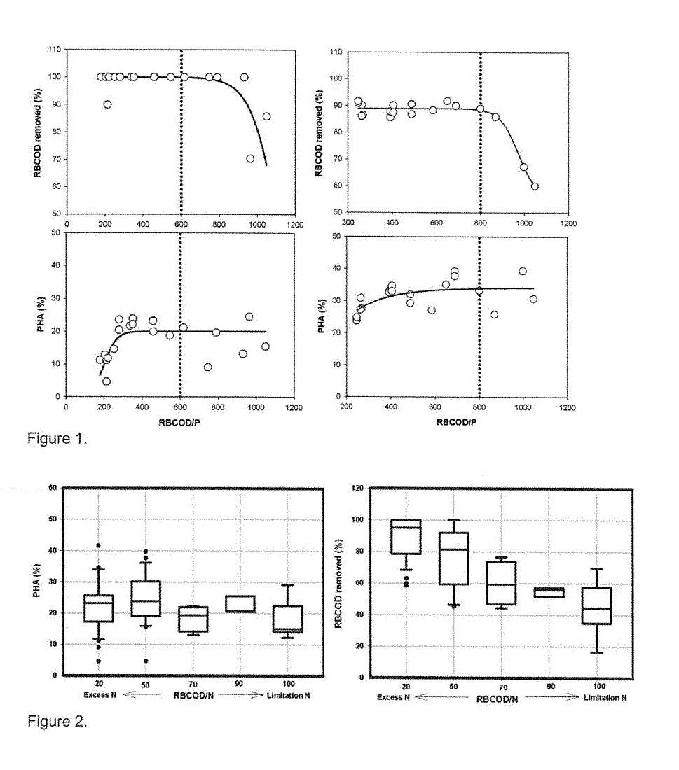

The PHA production within excess biomass was assessed under a range of influent RBCOD to phosphorus ratios at a process operating temperature of 37° C. Several RBCOD/P ratios were tested in the reactors treating fermented recycle paper mill process water (herein Paper Mill influent) containing RBCOD that was nominally 91 percent as VFA. In parallel, tests were performed with a reference feedstock (herein Reference influent) with RBCOD that was 100 percent VFA as acetic acid (FIG. 1). During these evaluations of the influence of RBCOD/P, the influent RBCOD/N was maintained constant at 30 g/g, which was a level experienced to be sufficient for the biomass nitrogen requirements in RBCOD removal.

Generally we found that RBCOD removal in the process was robust until the RBCOD/P was taken beyond 800 g/g, wherein either less reliable (Paper Mill influent) or a progressive decrease in treatment performance (Reference influent) was observed. At the other end of the scale, we found, that as the RBCOD/P increased above 200 g/g, an elevated PHA content in the biomass resulted. The Reference influent with 100 percent RBCOD as VFA gave on average consistently higher PHA content in the surplus biomass, suggesting a positive influence of VFA fraction of the RBCOD on the PHA production process. Thus we found that it is preferable, in general, to provide RBCOD to the process with higher VFA fraction wherein without stringent control of the organic specific loading, a process with continuous production of PHA was demonstrated with a PHA content in excess of 30% gPHA/gVSS.

Variability in the PHA content during the operation in the range of 800 RBCOD/P 200 g/g were later understood to be due to (at that time) less well-controlled fluctuations in the process biofilm bio-volume. For example, events of biofilm sloughing are understood to be concomitant with a decrease in biomass PHA content due to the associated increase in specific organic loading. The increase in specific organic loading was understood due to a rapid wash out of sloughed biofilm biomass due to the short process HRT. Conversely and anecdotally we also observed and later confirmed in Example 8, that selective retention of the RS-MLVSS generally resulted in enhanced biomass PHA content given the same loading and RBCOD/P conditions.

Phosphorus limitation sustains an elevated PHA content in the surplus biomass, while sustaining robust performance in RBCOD removal within a rather wide stable range of RBCOD/P from 200 to 800 g/g. This wide range is advantageous since in industrial process it may be more complicated or costly to implement a very stringent nutrient dosing strategy. Notwithstanding, we find based on the observed trends that a narrower range on average RBCOD/P between 300 and 800 g/g to be preferable for enhanced biomass PHA content.

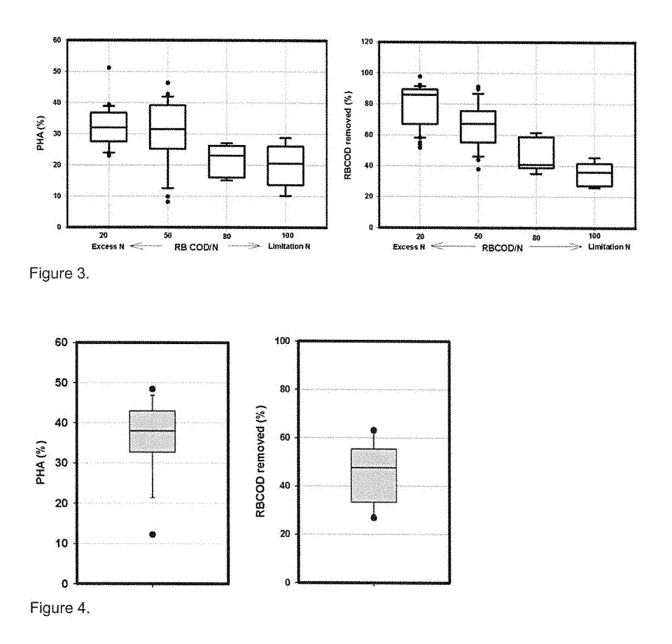

FIG. 2 and FIG. 3 report on findings of imposing N-limitation in combination with P-limitation also with process operating temperature of 37° C. We observed, for RBCOD/N greater than 50 g/g, a general decline in process RBCOD removal performance along side evidence to suggest, on average, a poorer PHA production outcome due to reduced biomass PHA content. Hence, double nutrient limitation did not promote conditions for improved PHA storage capacity. On the contrary, the highest PHA contents observed during these campaigns of testing were with an excess of influent N with respect to influent RBCOD.

Variability in PHA content during campaigns of RBCOD/N of 20 and 50 began to suggest an influence of specific organic loading on the resultant PHA content. We understand that maximal PHA content in the biomass was achieved when specific organic loading was reduced due to increased resident total biomass in the process.

It is known, to those experienced in biological treatment methods, nutrient deficient industrial wastewaters at times may require chemical addition of N and P to meet bacterial biological needs to metabolize and thereby remove soluble organic carbon. Addition of nutrients may involve process control measures and the chemical additions increase the cost of the treatment. Embodiments presented herein provide for a doubly beneficial outcome from the treatment of RBCOD by imposing P-limitation due to the reduced chemical demands and the production of a value-added raw material (PHA-rich-biomass) in a simple single bioprocess step. Nitrogen limitation does not appear to contribute to improve the performance of the process and in general we find that too little nitrogen detracts from both RBCOD treatment and PHA production performances. In a preferred embodiment of process operation with P-limitation, the average influent N is ideally kept as low as possible. RBCOD/N should be less than 70 and more preferably less than 50 g/g but more than 20 g/g.

### Example 2: Evaluation of Higher Process Operating Temperature

Methods and Materials.

A laboratory scale MBBR was operated with a reference RBCOD of acetic acid. The process performance was evaluated with fixed organic load (4 g-COD/L/d), hydraulic retention time HRT (1.5 h), and volumetric filling degree of 40% with AnoxKaldnes K3 carriers. Influent nitrogen levels were on average with RBCOD/N of 50 g/g and influent RBCOD/P was 800 g/g on average. The operating temperature was controlled at a set point temperature of 50° C. The reactor was continuously operated under the selected conditions for 120 days.

Results and Discussion.

Many industries treat and, in the best of possible circumstances, reuse contaminated process waters. Recycle paper mills are a practical example where process water temperatures are generally high and there exists a general economic benefit if these process waters can be treated without need for cooling. Biological processes can select for bacteria that are specialized to survive over a wide range of temperatures. Typically this selection is divided into ranges of temperatures for organisms that may be classified as psychrophiles 15° C.), mesophiles (20 to 45° C.) and thermophiles (>45° C.). Research literature concerning open (or mixed) culture production of PHA has been most extensively focused on laboratory trials with mesophiles. The ability to select for PHA producing organisms that are thermophiles while treating process waters or wastewaters has, to our knowledge, not been previously disclosed. We have assessed a potential for PHA production with thermophiles at 50° C. To our surprise we found that the embodiments of method and process could be applied with thermophiles and a continuous PHA production was demonstrated with elevated PHA contents between 30 and 50% gPHA/gVSS.

FIG. 4 depicts the process performance. The lower outliers in the PHA content and COD removal were associated with temperature transition in the reactor from being operated at 37° C. and then shifted to 50° C. Nevertheless, the thermal inactivation of mesophilic bacteria occurred quickly and stabile conditions were achieved with thermophiles capable to synthesize PHA.

Treatment efficiency under the thermophilic conditions was observed to be poorer than those reported in previous examples. In subsequent studies (Example 3) we observed that the performance in RBCOD removal correlates to enhancement in surplus biomass PHA content. We consider that further average improvements in performance to PHA production under thermophilic conditions are achievable by increasing the process biofilm bio-volume capacity and/or by increasing the SRT_{RS} of the RS-MLVSS.

### Example 3: Influence of Organic Carbon Limitation and Available Biofilm Biovolume

Materials and Methods.

An influent RBCOD comprising of 70% acetic acid and 30% propionic acid (COD basis) was treated in two parallel in MBBR reactors as described in previous examples. The process performance was evaluated with a fixed volumetric organic load (4 g-RBCOD/L/d), HRT (1.5 h), temperature (37° C.), and nutrient supply. Average influent RBCOD/N and RBCOD/P were 50 g/g and 700 g/g, respectively. The two MBBRs were with different volumetric filling degrees of AnoxKaldnes K5 carriers; one with 25% filling degree, and the other with 50% filling degree. The MBBRs were continuously operated and monitored over 150 days.

Results and Discussion.

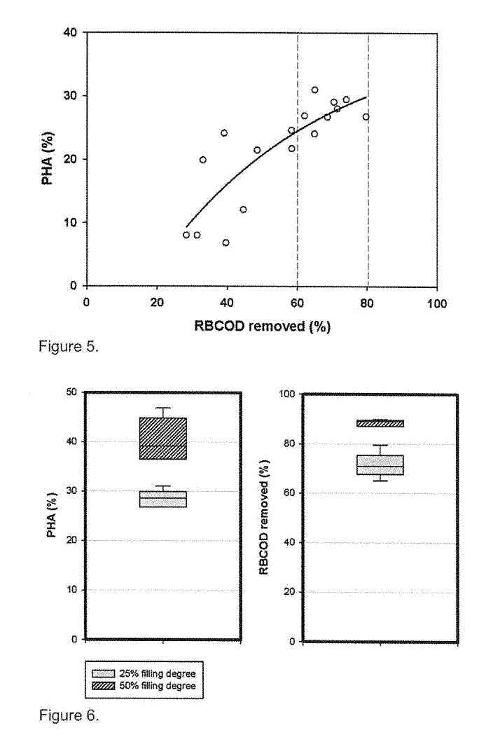

The PHA production performance was assessed with respect to residual RBCOD availability in the process. When the process performance in RBCOD removal decreases, the mixed liquor biomass grows in an environment with excess readily available carbon. One may interpret that under these conditions there is no advantage for survival of populations of species by storing PHA because substrate is readily available. Those bacteria that grow more quickly will become a more dominant part of the process ecosystem. FIG. 5 illustrates that as RBCOD removal performance increases, so does the PHA production performance. Therefore, the preferred embodiment for the PHA production process is a biological unit process that removes most if not all of the influent RBCOD.

We discovered the influence of specific organic loading by operating parallel MBBRs with different biocarrier filling degrees. Increasing the filling degree of AnoxKaldnes K5 carriers from 25 to 50% effectively doubled the available process biofilm bio-volume capacity. As the biofilm biomass grows it eventually becomes limited by the holding capacity provided by the filling degree of the specific biocarriers used. FIG. 6 illustrates the effect of increased process biofilm growth capacity on both the process performance in RBCOD removal and PHA production. The PHA production performance was enhanced when maximum influent RBCOD removal was achieved with an increased capacity for retaining biomass in the process due to more biocarriers. Thus from these experiments we found the role of decreasing specific organic loading rate towards enhancing PHA content of surplus biomass produced in a 1-step biofilm RBCOD treatment process.

### Example 4: Influence of Specific Organic Loading Rate

Materials and Methods.

Two MBBRs were run in parallel at laboratory scale as described above but with different biocarrier filling degrees (25% and 50%) as described for Example 3, with respect to the substrate treated and operational parameters. For the purpose of this experiment, the amount of biofilm biomass per reactor unit volume was quantified from microscopic evaluations of biofilm thickness using light microscopy SZ-CTV Olympus equipped with software NIS-Elements D 4.12.01.Ink and NIS-Elements Analysis D 4.12.01.Ink for image acquisition and analysis. Due to practical restrictions related to the small volume of the laboratory reactors, the nominal biofilm biomass content was evaluated in this way semi-quantitatively, and, thereby, non-destructively.

Based on image analysis outcomes, the amount of biofilm was related to the occupied protected surface area per reactor. The measured biofilm thickness was interpreted as a biofilm filling degree, being from 0-100% of the total protected surface area for biocarrier random grab samples.

The estimated biofilm filling degree was related to the total process protected surface area in the reactors, given by the known constant total number of the biocarriers in each reactor. The estimated values were an indicative relative quantification index to represent an average per carrier change in extant biofilm bio-volume. A higher bio-volume indicative index indicated for a greater amount of biofilm biomass at a given time point in the process operations. Given a similar indicative index value, the MBBR with higher carrier filling degree retained a greater total amount of biomass in the process.

Results and Discussion.

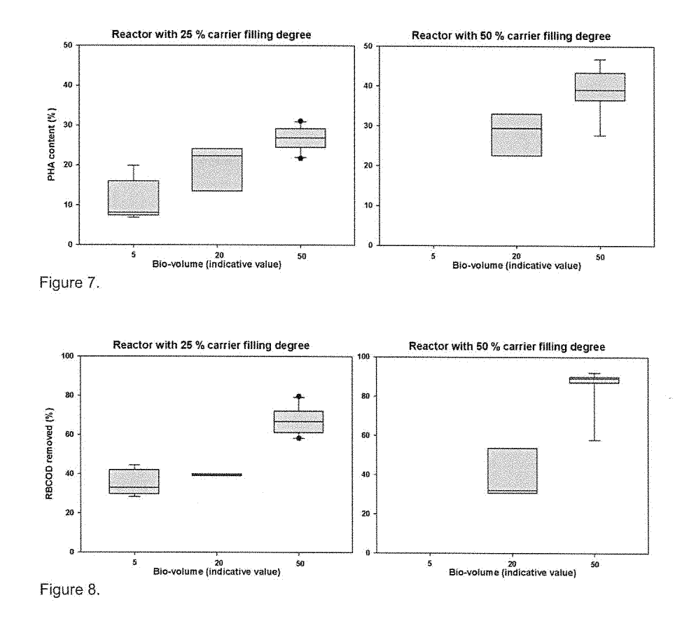

PHA production and COD removal efficiency in a continuous one-step process was found to be significantly influenced by the extant amount of biofilm (bio-volume) carried per carrier element in the process over the operating period (FIGS. 7 and 8). Variability in performance was observed due to periodic but substantial events of biofilm detachment. A sudden related decrease in the process specific organic loading rate was found to be associated with a general decrease in PHA production performance (lower surplus biomass PHA content). Given the same per carrier occupied biofilm bio-volume, a greater amount of total bio-volume (50% versus 25% carrier filling) resulted in a higher effluent biomass PHA content. Thus a greater process biomass inventory, meaning a lower specific organic loading rate, resulted in an enhanced biomass PHA content while feeding RBCOD under conditions of P-limitation.

Thus we found conditions for a continuous (or semi-continuous) PHA production process where P-limitation provides for enrichment of the PHA storing phenotype in the biomass, a stable lower specific organic loading rate generally achieves an enhanced PHA content in the surplus biomass.

The increased capacity for biofilm bio-volume for the reactor with 50% carrier filling degree meant for a much thinner well-distributed biofilm compared to reactor with 25% filling degree. In the case of the 25% filling, carriers became filled with biofilm to the point where effectively the process biomass bio-volume (biofilm surface area to a maximum biofilm depth of 0.25 mm) decreased. Under these limiting conditions, we also observed a general loss in performance because the intended protected surface area was no longer being utilized as the K5 carrier design was intended. It is known that substrate and oxygen diffusion into the biofilm becomes heavily restricted with an increasing biofilm thickness causing the build up of non-participating biomass in the process.

Performance of a continuous biofilm system for PHA production is primarily dependent upon biofilm development. The system should be controlled to assure the consistent production of an active biomass enriched with PHA storing bacteria based on P limitation. An active biofilm maintains an elevated kinetic rate of COD removal, which was the case for the reactor with a higher surface area and a thinner, more active biofilm where conditions were found that resulted in enhanced PHA production performance.

### Example 5: Process Examples

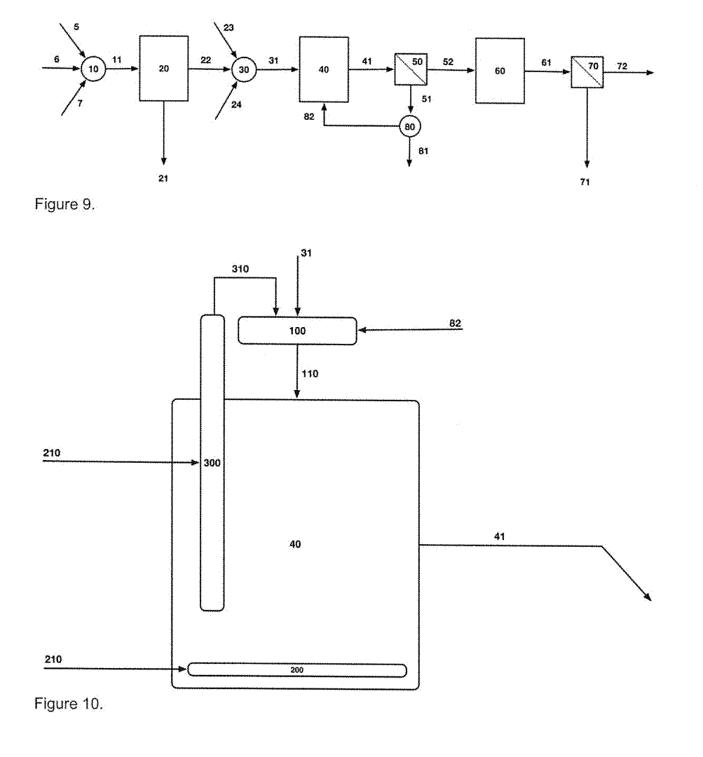

Without limitation, an illustrative process schematic diagram is presented in FIG. 9. In this example, a number of process water or wastewater effluent streams (5, 6 and 7) are combined (10) and disposed (11) to an optional (if necessary) anaerobic unit process (20). The unit process 20 converts non-RBCOD into RBCOD (22). The surplus biomass from the fermentation process (20) may be exported (21) and managed as an organic sludge. The RBCOD rich effluent (22) may be combined (30) with other process waters, wastewaters and/or chemicals (23 and 24) in order to create an influent (31) satisfying the RBCOD/N and RBCOD/P fora continuous or semi-continuous biofilm PHA production process (40). Control of stream combinations in 30 may be given based on feedback in the measured performance of 40 in PHA production and RBCOD removal. Most preferably the influent RBCOD stream (31) is a VFA rich stream.

The unit process 40 is an aerobic or anoxic biofilm process wherein biomass is principally retained in 40 attached to the available biofilm surfaces and a surplus biofilm biomass is produced by detachment from the biofilm surfaces. Effluent biomass is with enhanced (greater than 30% gPHA/gVSS) PHA content. The detached biofilm biomass aggregates are to dominate the reactor MLVSS as readily separable MLVSS (RS-MLVSS) in the effluent mixed liquor (41), containing significantly reduced levels of RBCOD with respect to the influent (31). Most of the influent RBCOD is to be removed in the process. Effluent RS-MLVSS dominates over free-living MLVSS (FL-MLVSS). Effluent mixed liquor is disposed to a separation process (50) where RS-MLVSS is selectively retained and FL-MLVSS is washed out. The retained RS-MLVSS comprising PHA-rich-biomass may be concentrated and separated from the main hydraulic flow (51). At least a fraction of the concentrated and separated RS-MLVSS may be returned (82) to the PHA production process (40) as a means to control SRT_{RS} of the RS-MLVSS to be greater than the HRT of the biofilm bioprocess (40) but preferably less than 1 day. Otherwise, the concentrated RS-MLVSS is exported from the process (81) and is a concentrated stream of biomass with enhanced PHA content as a value added residual from the wastewater treatment.

Given that Q is the influent volumetric flow rate (m³/hr), V is tank process volume (m³), and X_{RS} is the RS-MLVSS concentration (g/m³), then with reference numbering in FIG. 9, one can define the following:

$${HRT}_{40} = \frac{{V}_{40}}{{Q}_{31}}$$

$${SRT}_{RS .40} = \frac{{V}_{40} {X}_{RS 40}}{{Q}_{81} {X}_{RS .81} + {Q}_{52} {X}_{RS .52}}$$

where HRT₄₀ is the hydraulic retention time for the biofilm process (40) and SRT_{RS.40} is the solids retention time for the RS-MLVSS that is produced by the biofilm process (40). Under the conditions provided in embodiments for P-limitation and RBCOD removal in the biofilm process, we found that by maintaining SRT_{RS.40}>HRT₄₀, the PHA-content of the MLVSS became enhanced. We consider from findings in example 8 that maintaining the process 40 average MLVSS concentration to be at least 10% greater than the average MLVSS established for conditions of no selective retention of RS-MLVSS (SRT_{RS.40}≈HRT₄₀), provides a practical criterion of maintaining SRT_{RS.40}>HRT₄₀).

The mixed liquor after selective RS-MLVSS separation and recirculation may be directed (52) to any number of optional (if necessary) downstream unit processes (60) where further objectives of water quality improvement may be achieved. Without limitation, the unit process 60 may include biological and/or physic-chemical treatment methods. The further objectives of water quality improvement could for example be related to reduction of residual RBCOD and other non-RBCOD forms of organic content that remain after the unit process 40. Similarly the effluent from 60 (61) may be directed to a separation step (70) where suspended solids are removed (71) and a treated final effluent meeting specified water quality demands (71) is produced.

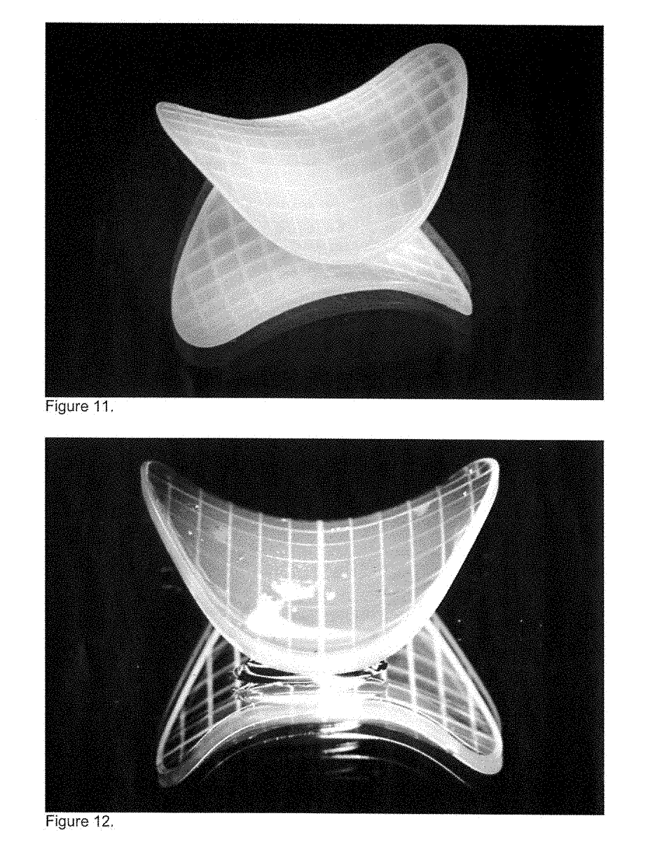

The unit process 40 is a biofilm process and could, for example, be an MBBR unit process (FIG. 10). This is an aerobic unit process with oxygen provided from aeration blowers that are directed to the bioreactor (210). The reactor is a completely mixed vessel, containing well-mixed biofilm carriers supplying process biofilm bio-volume, with mixing and aeration provided for by a distribution of fine or coarse bubble diffusers (200). MBBR biocarriers that are also well-mixed within the process volume (40) provide capacity of biofilm bio-volume. Biocarriers retain the major fraction of the resident biomass in the process volume, and the biocarriers themselves are retained in the process volume by means of sieve structures in advance of the effluent outlets. Detached biofilm enters the process mixed-liquor and becomes readily separable MLVSS (RS-MLVSS). Some or all of the effluent RS-MLVSS may be selectively harvested from the process effluent (41). Selectively returning a fraction of the effluent RS-MLVSS (82) creates for conditions wherein the RS-MLVSS retention time (SRT_{RS}) is increased from the nominal RS-MLVSS retention time that is equal to the process HRT. The influent (31) flow rate establishes a sufficiently short process HRT whereby the detached biofilm biomass aggregates in the mixed-liquor (or RS-MLVSS) dominate over the mass of free-living bacteria in the mixed liquor (or FL-MLVSS). The HRT is furthermore sufficiently long to reduce the influent RBCOD to as great an extent as possible. The organic loading rate to the process may be typically designed to be between 1 and 15 gCOD/L/d. The process biomass may be repeatedly stimulated by an elevated RBCOD concentration by means of an influent contact (stimulation) zone (100). Mixed liquor containing process biomass are brought by means of an airlift (300) and directed (310) to the stimulation zone (100) with a repeated frequency that may be controlled by the number of airlifts and volumetric flow produced by the airlift operation. The stimulation zone does not need to be aerated and the zone establishes a nominal period of time in which the biofilm experience RBCOD concentrations that are generally to be higher than 100 mg/L and less than 2000 mg/L on average. The blend of influent and airlift recirculation mixed liquor are directed to the main vessel (40) wherein, a high biomass respiration rate capacity is generally maintained by the repeated and periodic events of stimulation for at least a fraction of the biomass at any given time. Selectively retained RS-MLVSS may be recirculated and maintained in the process (82), in order to increase SRT_{RS} of the RS-MLVSS to be greater than the process HRT. Notwithstanding, the SRT_{BB} of the biofilm biomass in the process is generally uncontrolled but is understood by those knowledgeable in biofilm process design to be significantly longer than the process HRT.

### Example 6: Concept of Biofilm Bio-Volume Capacity

The AnoxKaldnes Z-MBBR biocarrier element provides illustrative example of the concept of biofilm process bio-volume capacity. The Z-MBBR biocarriers are saddle shaped wafers with a matrix of wells of defined depth on both surfaces (FIG. 11). The projected surface area (perpendicular to the saddle surface), of a biofilm growing in each well, does not change with increasing depth of biofilm. Biofilm growth is generally protected as long as the biofilm growth does not protrude out of the confines of protection afforded by each respective well. The biofilm fragments that grow beyond the well depth will be sheared and become detached due to frequent contact with other carriers within the MBBR process volume (FIG. 12). The aerobic bio-volume is defined by the biofilm surface area down to the penetration depth of dissolved oxygen. Nominally this depth is anticipated to be approximately 0.2 mm. Notwithstanding, factors such as biofilm density and dissolved oxygen concentration may be expected to influence this depth of dissolved oxygen penetration.

Given the example of Z-MBBR utilizing carriers with a 0.2 mm well depth, of the Z-MBBR, the process biofilm bio-volume capacity may therefore be defined by the number of carriers per unit volume, multiplied by the number of wells per carrier, multiplied by the volume per well.

### Example 7: Influences of P-Limitation and Periodic Repeated Biomass Stimulation

Materials and Methods.

Two MBBRs of 6.2 L working volume were run in parallel at laboratory scale with 1700 Anoxkaldnes Z-carriers (filling degrees 45%) with 0.4 mm well-depth. The reactors were inoculated with biomass as activated sludge collected from the Kallby/Lund municipality WWTP, Sweden. The MBBRs were operated with a reference RBCOD of acetic acid. Additions of bio-available forms of phosphorus (P), with KH₂PO₄ were made to the influent flow to adjust to selected influent RBCOD/P ratios. Bioavailable nitrogen (N) was added with NH₄Cl in order to maintain a constant RBCOD/N of 40. Trace elements were also added in order to ensure that micronutrients for the biological process were not limiting. The composition of the trace nutrient solution and additions are described elsewhere (Bengtsson, 2009, Biotechnology and Bioengineering, 104(4):698-708). Peptone and yeast extract were also added to the influent flow but this addition accounted for ≦2%, of the total COD in the influent.

Aeration by means of compressed air through a diffuser plate at the bottom of the reactor provided mixing energy and maintained the process with dissolved oxygen between 3 and 5 mg-O₂/L. Reactors were jacketed and heating water was recirculated from a regulated bath to the reactor jacket so as to maintain the treatment process temperature at 37° C. Reactors were operated with influent flow rate for a 1-hour HRT and with an average organic loading rate (OLR) of 6 gCOD/L/d. Both reactors were operated similarly with constant HRT and the same average OLR, with the exception that the OLR in was constant in one MBBR (reference, or R-MBBR) and it was varying in time in the other MBBR (dynamic, or D-MBBR).

In the D-MBBR the OLR was split between a constant flow equivalent to 90% of the total OLR and the remaining 10% of the OLR was supplied through a periodic input of concentrated influent. The aliquots or pulses of influent were added using a diaphragm pump (5 mL pumped in 1 min every 8 hours). The D-MBBR mimicked in time at laboratory scale, the process configuration depicted in space in FIG. 10 (elements 100 and 40), wherein, the biomass were periodically and repeatedly disposed to a stimulation zone, and otherwise were maintained with low background RBCOD concentrations due to removal of COD from biological activity.

The effluent biomass or RS-MLVSS was separated from the effluent and composite samples collected over 20 minutes were used for quantitative assessment of the biomass PHA content. PHA content was determined by thermogravimetric analysis of dried biomass samples (see US 2013/0203954 A1). The fate of COD, nitrogen and phosphorus across the MBBRs were monitored and mass balances were performed. Conversion and removal rates were estimated based on these mass balance considerations.

In order to evaluate for an influence of D-MBBR versus R-MBBR operating conditions, batch stress tests were conducted to compare the RBCOD, N and P maximum uptake rates. For such a stress test, influent flow to the MBBRs was shut off and a defined aliquot of substrate to reach a nominal peak RBCOD concentration of 200 mgCOD/L was dosed to the reactor now operating under batch conditions. Samples from the reactor at selected times over approximately two hours were used to follow the trends and removal rates of added COD, N, and P. Maximum rates of RBCOD, N, and P removal were compared to the steady state removal rates based on mass balances in continuous operation at 1 hour HRT and 6 g/L/d OLR.

The PHA content of the effluent MLVSS in continuous operation was compared to the PHA accumulation potential (PAP) of the effluent biomass. PAP tests were run by disposing the effluent biomass to a period of sustained feast for a period of 5 to 10 hours. From trends of increase in PHA content of the biomass in time, the maximum PHA content reached by the biomass was interpreted as the biomass extant PHA accumulation potential. These accumulation experiments resemble those that are conducted as Step 2 in a so-called two-step PHA production process, wherein surplus biomass is collected from Step 1 and then disposed to conditions of PHA accumulation in Step 2. The PHA accumulation experiments were run respectively for R-MBBR and D-MBBR effluent MLVSS in 2 parallel fed-batch feed-on-demand respiration controlled bioreactors following methods and equipment that has been previously described (Valentino et al. (2015). Water Research. 77, 49-63). Two glass-jacketed reactors with a working volume of 2 L were used. The batch accumulation reactors were maintained at 37° C. and aeration was provided at a flowrate of 1 L/min. Accumulation substrate consisted of 100:1:0.05 (COD:N:P) at pH 7, where the COD was sodium acetate. The intermittent pulse-wise substrate addition was set to give a dose-peak substrate stimulation concentration of 100 mg-COD/L in the reactor.

For the PAP experiments, about 20-30 L MBBR effluent RS-MLVSS biomass was collected and left to settle at 4-5° C. overnight. The supernatant was decanted and the remaining volume was centrifuged (3500×g for 5 minutes). The concentrated biomass was resuspended in tap water to a concentration of between 500 and 700 mg-TSS/L. The accumulation reactors were aerated to steady state conditions of DO and pH before starting the fed-batch PAP accumulations. Experiments were terminated with an onset of reduced respiration response to substrate additions. Samples of mixed liquor from the accumulation reactors were taken at selected times and the trends in accumulation were evaluated based on thermogravimetric analysis (TGA) of the biomass and COD, N, and P analyses of the reactor water quality.

Mass balances for the respective MBBR process operations were performed over several weeks with a nominal operating RBCOD/P of 200 (P-balanced loading). These benchmark conditions suggested a slight excess of P-supply to the process. Based on the mass balance evaluations with benchmark operating conditions, the P requirements of the process were estimated and a small step in P-limitation was applied to both the R-MBBR and D-MBBR, respectively. The R-MBBR was subsequently operated with RBCOD/P of 326 g/g and the D-MBBR was operated with a RBCOD/P of 266 g/g.

Effluent RS-MLVSS with respect to the effluent MLVSS concentrations were estimated based on a simple test of gravity settling. For this evaluation, effluent was collected (750 mL) and the well-mixed sample was split evenly between three 250 mL graduated cylinders. Immediately after decanting the well-mixed sample into respective volumetric cylinders, a 3 mL sample was taken from the upper 50 mL volume for estimation of the volatile (organic) suspended solids, measured as COD. The organic suspended solids concentrations were estimated as the difference between the sample total COD minus the sample soluble COD levels. Soluble COD levels were measured after sample filtration following Standard Methods. After two hours, another sample was similarly taken and analysed. The settleable suspended solids or RS-MLVSS levels in the MBBR effluent were evaluated for all three graduated cylinders and thereby in triplicate for each effluent grab sample.

Results and Discussion.

For both R- and D-MBBRs under both P balanced and P limiting loading phases, the removal of COD was nominally 88% and VFA removal was above 90% on average. Thus, independent of either P balanced or after the step to P limiting operating conditions for both reactors, the MBBRs exhibited similar and robust removal efficiencies and steady state uptake rates of influent RBCOD, N and P. Based on the sedimentation measurements, most of the effluent biomass MLVSS (79±11%) was estimated to be readily separable MLVSS, or RS-MLVSS for these operating conditions.

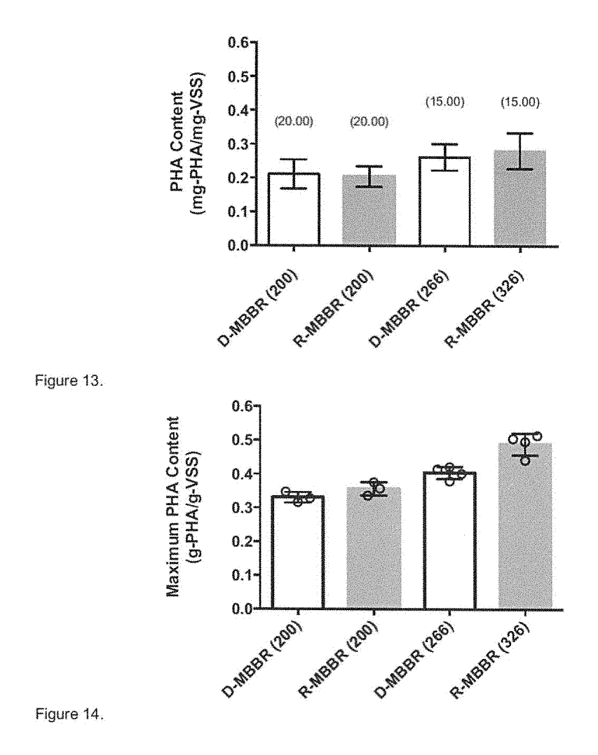

During the P balanced phase (over 64 days), the effluent biomass was with an average PHA content of 0.21 gPHA/gVSS and these levels increased to an average level of 0.28 g-PHA/g-VSS after shifting to P-limiting conditions (over 36 days). FIG. 13 reports on the PHA content from R- and D-MBBRs for the benchmark P-balanced (RBCOD/P=200 g/g), and for the period of P-limited loadings (RBCOD/P=326 and 266 for R-MBBR and D-MBBR, respectively). In FIG. 13 the average value is the height of the bars and the number of samples over 64 days and 36 days of operation at the respective RBCOD/Ps are shown in brackets. Error bars are the standard deviation of the mean values. An increase shift on average towards increased effluent biomass PHA content was stimulated by a relatively small increase in RBCOD/P from nutrient balanced conditions (RBCOD/P 200 g/g).

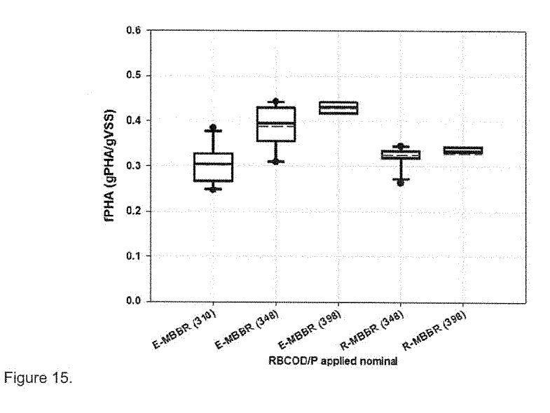

FIG. 14 shows that the PHA accumulation potential (PAP) of the biomass increased from 0.3 (D-MBBR) and 0.36 (R-MBBR) to 0.4 (D-MBBR) and 0.49 (R-MBBR) gPHA/gVSS after the small shift towards P limitation. On average, the MBBR effluent MLVSS produced with P limitation (RBCOD/P>200) demonstrated an enhanced PAP than the effluent MLVSS biomass that was produced during the P balanced phase. Furthermore, the reference reactor showed higher PAP on average wherein this MBBR was operated with RBCOD/P>300 g/g. In FIG. 14, the open circles represent the measured PAP from respective PHA accumulation experiments from which an average biomass PAP was calculated. Error bars express the standard deviation of the mean value.

During both P loading phases (RBCOD/P=200 and RBCOD/P>200), the D-MBBR effluent biomass RS-MLVSS expressed on average a higher fraction (64-65%) of the estimated PAP compared to the reference reactor (57%). Relatively speaking, the dynamic stimulation of the biomass was observed to exert a positive influence on the expression of PHA accumulation potential. The R-MBBR did maintain a higher absolute PAP on average, which does fit with expectation based on the difference in applied RBCOD/P.

Since the RBCOD removal efficiencies of the R- and D-MBBRs were similar, the steady state RBCOD removal rates were estimated to be the same (3.7 mgCOD/L/min) during balanced conditions of operation with RBCOD/P equal to 200 g/g. From batch experiments the process capacity for COD removal was estimated to be similar but on average slightly higher for the D-MBBR (3.9±0.7 versus 4.3±0.5 mgCOD/L/min for the R- and D-MBBRs, respectively). Under conditions of P-limitation (RBCOD/P>200), the steady state removal rates were slightly lower (3.4 and 3.5 mgCOD/L/min for the R- and D-MBBRs). However, we found that the capacity for COD removal was significantly higher for the D-MBBR for P-limiting operating conditions (3.3±0.7 versus 5.5±1.0 mgCOD/L/min for the R- and D-MBBRs). Therefore, repetitive and repeated stimulation of the reactor biomass was found to sustain a higher process capacity for COD removal.

From these experiments we experienced that application of an RBCOD/P greater than 200 did increase the PAP of the biomass but the expression of this PAP in the effluent biomass MLVSS was less than expected.

### Example 8: Influence of P-Limitation and Increased RS-MLVSS SRT with Respect to HRT

Materials and Methods.

The two MBBRs from Example 7 were operated in parallel with the exception from Example 7 that both MBBRs were maintained with a steady state influent organic loading as described for the R-MBBR. Both reactors were subject to the same flows and operating influent conditions as reported for Example 7 and the R-MBBR. However, in one of the MBBRs the readily separable MLVSS (RS-MLVSS) was selectively retained in one of the MBBRs in order to create conditions in which the RS-MLVSS SRT_{RS} was selectively made to be greater than the process hydraulic retention time (HRT). RS-MLVSS was selectively retained in one of the MBBRs by providing a quiescent zone in the reactor volume adjacent to the effluent outlet. This quiescent zone permitted for more readily settleable volatile suspended solids to remain entrained for a longer period of time in the otherwise well mixed bioreactor volume. Therefore, in this example we maintained one reference MBBR or R-MBBR as before in Example 7, and a second reactor identical to the R-MBBR except that the RS-MLVSS was selectively with an extended SRT_{RS} (E-MBBR). The condition of RS-MLVSS SRT_{RS} greater than HRT was applied in the E-MBBR and the condition of RS-MLVSS SRT_{RS} equal to HRT was maintained in the R-MBBR. The influence of similar increasing shifts of RBCOD/P towards enhancing the effluent MLVSS PHA content were evaluated for both the R-MBBR and the E-MBBR, respectively.

Grab samples of the MBBR mixed liquor were obtained routinely over the course of steady state operations for periods with RBCOD/P equal to 324, 348 and 398 g/g. The MLVSS concentrations of the MBBRs were estimated as well as the PHA content of the biomass. The MLVSS concentrations were estimated based on COD measurements and mass balance considerations.

Results and Discussion.

From Example 7 it was observed that a modest but small definitive step of increase in RBCOD/P over 200 g/g created for operating conditions with a P limitation and that this step increase in RBCOD/P similarly promoted increased PHA accumulating potential (or improved enrichment) of the biomass comprising the effluent MLVSS in both the R- and D-MBBRs. It should however be noted that even under balanced nutrient loading conditions of RBCOD/P equal to 200, both reactors described in Example 7 produced an effluent biomass enriched with PAP, or PHA accumulation potential (PAP>30% gPHA/gVSS). Increasing the RBCOD/P did not improve the relative expression of the extant PAP for the effluent MLVSS. Our objectives have been to establish process and methods towards producing a biomass with enhanced PHA content (>30% gPHA/gVSS) in just one bioreactor step. The results of Example 7, wherein the RS-MLVSS SRT_{RS} was equal to the process HRT, suggested that increasing RBCOD/P to be greater than 300 can result in the production of a biomass with increased enrichment for PHA accumulation potential. Notwithstanding, we wished to find methods that would express this potential in just one step so as to produce a biomass with enhanced PHA content in this one step.

FIG. 15 for the present example describes our findings that when at least some of the effluent RS-MLVSS is selectively retained such that RS-MLVSS SRT_{RS} becomes longer than the process HRT, enhancement of the effluent biomass PHA content was achieved with levels of PHA content exceeding 30% gPHA/gVSS and preferably 40% gPHA/gVSS. During this period of operation with shifts in applied RBCOD/P, the RBCOD removal efficiency was on average 92% and 91% for the R- and E-MBBR respectively.

Over the course of the monitoring period the MLVSS levels in the R-MBBR and E-MBBR were on average 83±9 and 125±79 mg/L, respectively. Notwithstanding that the MLVSS levels in the E-MBBR were more variable in nature, based on the average value of MLVSS, these results suggest an influence of the settling zone in the E-MBBR that permitted to extend the RS-MLVSS SRT_{RS} to be in the order of 1.4 times the MBBR HRT wherein the HRT was nominally 1 hour in these experiments. Thus, conditions of SRT_{RS} greater than HRT are satisfied when the effluent MLVSS concentration with RS-MLVSS selective retention is measurably greater on average than the MLVSS concentration without RS-MLVSS retention. Absence of selective RS-MLVSS retention represents conventional MBBR operating design as is understood for those skilled in the art of MBBR process designs and configurations. Based on the variability of the MLVSS concentration with the conventional R-MBBR operation, E-MBBR operation should establish at least 10% higher MLVSS levels than those attained on average in a conventional R-MBBR operation mode.

The present invention may, of course, be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

1-100. (canceled)

101. A method of treating a wastewater stream containing readily biodegradable chemical oxygen demand (RBCOD) and phosphorus (P) in a moving bed bioreactor (MBBR) wherein the MBBR includes biomass attached to biomass carriers and detached biomass fragments referred to a readily separable mixed liquor volatile suspended solids (RS-MLVSS) and wherein the method produces biomass with an enhanced polyhydroxyalkanoates (PHA) content; the method comprising:
directing the wastewater influent into the MBBR and maintaining the RBCOD/P ratio in the wastewater influent directed into the MBBR at 200-800 g/g;
biologically treating the wastewater in the MBBR and removing RBCOD from the wastewater and producing an MBBR effluent containing the RS-MLVSS;
maintaining the sludge retention time (SRT) of the RS-MLVSS such that the SRT of the RS-MLVSS is greater than the hydraulic retention time (HRT) of the MBBR;
separating the RS-MLVSS from the MBBR effluent into first and second streams and recycling the first stream of separated RS-MLVSS back to the MBBR;
harvesting the detached biomass fragments from the second RS-MLVSS stream; and
wherein by maintaining said RBCOD-P ratio and recycling said first stream of the RS-MLVSS, on average, produces a biomass with a PHA content that is greater than 30% g PHA/gvss.

102. The method of claim 101, wherein the SRT of the RS-MLVSS is controlled to be less than 1 day.

103. The method of claim 101, wherein the MBBR is operated at a temperature between 20 and 60° C.

104. The method of claim 101 wherein the wastewater stream contains nitrogen (N) and the method includes controlling the wastewater influent directed to the MBBR by maintaining the RBCOD/N ratio in the wastewater influent to between 20 and 70 g/g.

105. The method of claim 101, wherein the wastewater influent is exposed to at least a fraction of a process biomass at any given time in a stimulating zone upstream of the MBBR.

106. The method of claim 101, wherein at least a fraction of the biomass in the MBBR is recirculated to and from a stimulation zone and the RBCOD concentration in the stimulation zone is maintained on average to be greater than 100 mgCOD/L.

107. The method of claim 101 wherein maintaining an average RBCOD/P ratio of between 200 and 800 gig is accomplished by controlling the flow rate of the wastewater influent into the MBBR.

108. The method of claim 101 including controlling the wastewater influent flow rate to establish a hydraulic retention time (HRT) in the MBBR such that detached biofilm are aggregates in the RS-MLVSS and these aggregates dominate over the mass of free living bacteria in the wastewater contained in the MBBR.

109. The method of claim 101 including transferring the biofilm carriers and the biomass thereon from the MBBR to a stimulating zone that lies outside of the MBBR; stimulating the biomass on the biofilm carriers in the stimulating zone by exposing the biomass to RBCOD in the stimulating zone; and continuing to transfer the biofilm carriers back and forth between the MBBR and the stimulating zone.

110. The method of claim 101 wherein maintaining the RBCOD/P ratio between 200 and 800 g/g comprises selectively combining a series of different wastewater streams to form the wastewater influent directed into the MBBR.

111. A method for producing a biomass with enhanced polyhydroxyalkanoate (PHA) content, the method comprising:
providing at least one feed stream, the feed stream comprising RBCOD;
biologically treating the feed stream and removing RBCOD from the feed stream while producing a biomass with a PHA content that is greater than 30% g PHA/gVSS by:
directing the feed stream to a moving bed bioreactor (MBBR), the MBBR comprising a stimulating zone and a main vessel and further comprising biomass carriers, wherein the feed stream is directed into the MBBR by way of the stimulating zone such that the RBCOD concentrations in the stimulating zone are, on average, higher than 100 mg/L and less than 2000 mg/L;
mixing the biofilm carriers within the MBBR, such that the carriers are mixed between the stimulating zone and the main vessel;
growing a biofilm on the biofilm carriers, the biofilm biologically removing RBCOD from the feed stream and producing a treated effluent;
wherein some of the biofilm becomes detatched from the biofilm carriers to produce detached biomass fragments referred to as readily separable mixed liquor volatile suspended solids (RS-MLVSS);
directing the treated effluent containing RS-MLVSS from the MBBR through a sieve, wherein the sieve allows the treated effluent to flow through the sieve while retaining the biocarriers in the MBBR; and
selectively recycling at least a fraction of the RS-MLVSS from the treated effluent to the MBBR;
wherein the retention of the biocarriers in the MBBR and the recycling of at least a fraction of the RS-MLVSS results in biofilm and RS-MLVSS having a solids retention time (SRT) that is greater than the hydraulic retention time (HRT) of the MBBR; and
harvesting at least a fraction of the RS-MLVSS from the treated effluent.

112. The method of claim 111, wherein selectively recycling at least a fraction of the RS-MLVSS from the treated effluent to the MBBR and harvesting at least a fraction of the RS-MLVSS from the treated effluent include:
directing the treated effluent containing RS-MLVSS is to a separator; and
separating the RS-MLVSS from the treated effluent to form a separated effluent.

113. The method of claim 112, further comprising
directing the separated effluent to a filter; and
filtering the separated effluent to remove suspended solids and producing a reject stream and a permeate.

114. The method of claim 111, wherein the at least one feed stream further comprises nitrogen (N) and wherein providing at least one feed stream, the feed stream comprising RBCOD, comprises mixing at least two wastewater streams to provide a feed stream with an RBCOD/N ratio between 20 gRBCOD/gN to 70 gRBCOD/gN.

115. The method of claim 111, wherein the at least one feed stream further comprises phosphorus (P) and wherein providing at least one feed stream, the feed stream comprising RBCOD, comprises mixing at least two wastewater streams to provide a feed stream with an RBCOD/P ratio between 200 gRBCOD/gP to 800 gRBCOD/gP.

116. The method of claim 111, wherein providing at least one feed stream, the feed stream comprising RBCOD, comprises providing a feed stream wherein at least 50% of the RBCOD is present as volatile fatty acids (VFAs).

117. The method of claim 111, wherein providing at least one feed stream, the feed stream comprising RBCOD comprises:
providing a wastewater stream comprising RBCOD, wherein less than 50% of the RBCOD is present as volatile fatty acids (VFAs);
directing the wastewater stream to a fermenter;
fermenting the wastewater stream in the fermenter to convert non-VFA RBCOD to UFA RBCOD and producing a feed stream comprising RBCOD wherein at least 50% of the RBCOD is present as VFAs.

118. The method of claim 117, wherein providing the wastewater stream further comprises mixing at least two waste streams together to form a single wastewater stream comprising RBCOD wherein less than 50% of the RBCOD is present as volatile fatty acids.
